SEQUENCE LISTING (39 sequences)

SEQ ID NO: 1 - protein, 5 aa, synthetic construct
VDNAL

SEQ ID NO: 2 - protein, 20 aa, synthetic construct
VDNALQSGNSQESVTEQDSK

SEQ ID NO: 3 - protein, 33 aa, synthetic construct
DNHYGSGSYYYYQYGMDVWGQGTTVTVSSASTK

SEQ ID NO: 4 - protein, 21 aa, synthetic construct
  MOD_RES 2: note = Glycoylated
HTFSGVASVESSSGEAFHVGK

SEQ ID NO: 5 - protein, 10 aa, synthetic construct
  MOD_RES 4: note = Glycosylated
NQVSLTCLVK

SEQ ID NO: 6 - protein, 10 aa, synthetic construct
  MOD_RES 4: note = Glycosylated
NQVNLTCLVK

SEQ ID NO: 7 - protein, 11 aa, synthetic construct
  MOD_RES 5: note = Glycosylated
VTSPNITVTLK

SEQ ID NO: 8 - protein, 11 aa, synthetic construct
  MOD_RES 7: note = Glycosylated
KGFIISNATYK

SEQ ID NO: 9 - protein, 8 aa, synthetic construct
  MOD_RES 4: note = Glycosylated
LVLNCTAR

SEQ ID NO: 10 - protein, 7 aa, synthetic construct
  MOD_RES 2: note = Glycosylated
KNSTFVR

SEQ ID NO: 11 - protein, 14 aa, synthetic construct
  MOD_RES 5: note = Glycosylated
SVIPSDGPSVACVK

SEQ ID NO: 12 - protein, 14 aa, synthetic construct
  MOD_RES 12: note = Glycosylated
CGLVPVLAENYNKT

SEQ ID NO: 13 - protein, 5 aa, synthetic construct
  MOD_RES 2: note = Glycosylated
INHCR

SEQ ID NO: 14 - protein, 21 aa, synthetic construct
  MOD_RES 9: note = Glycosylated
QQQHLFGSNVTDCSGNFCLFR

SEQ ID NO: 15 - protein, 32 aa, synthetic construct
  MOD_RES 28: note = Glycosylated
RPTGEVYDIEIDTLETTCHVLDPTPLANCSVR

SEQ ID NO: 16 - protein, 15 aa, synthetic construct
  MOD_RES 12: note = Glycosylated
LCPDCPLLAPLNDSR

SEQ ID NO: 17 - protein, 28 aa, synthetic construct
  MOD_RES 17: note = Glycosylated
VVHAVEVALATFNAESNGSYLQLVEISR

SEQ ID NO: 18 - protein, 61 aa, synthetic construct
  MOD_RES 26: note = Glycosylated
  MOD_RES 35: note = Glycosylated
  MOD_RES 37: note = Glycosylated
  MOD_RES 51: note = Glycosylated
VTCTLFQTQPVIPQPQPDGAEAEAPSAVPDAAGPTPSAAGPPVASVVVGPSVVAVPLPLHR

SEQ ID NO: 19 - protein, 15 aa, synthetic construct
  MOD_RES 1: note = Glycosylated
  MOD_RES 8: note = Glycosylated
TPIVGQPSIPGGPVR

SEQ ID NO: 20 - protein, 54 aa, synthetic construct
  MOD_RES 37: note = Glycosylated
MNVLLLLVLCTLAMGCGATSPPQPAARPSSLLSLDCNSSYVLDIANDILQDINR

SEQ ID NO: 21 - protein, 39 aa, synthetic construct
  MOD_RES 9: note = Glycosylated
RIFYLPAYNCTLRPVSQSAIIMTCPDCPSTSPYDLSNPR

SEQ ID NO: 22 - protein, 15 aa, synthetic construct
  MOD_RES 3: note = Glycosylated
GENATVNQRPANPSK

SEQ ID NO: 23 - protein, 17 aa, synthetic construct
  MOD_RES 9: note = Glycosylated
  MOD_RES 12: note = Glycosylated
TEELQQQNTAPTNSPTK

SEQ ID NO: 24 - protein, 6 aa, synthetic construct
NSTFVR

SEQ ID NO: 25 - protein, 10 aa, synthetic construct
GFIISNATYK

SEQ ID NO: 26 - protein, 9 aa, synthetic construct
EEQYNSTYR

SEQ ID NO: 27 - protein, 7 aa, synthetic construct
TKPREEQ

SEQ ID NO: 28 - protein, 14 aa, synthetic construct
  MOD_RES 5: note = Glycosylated
DSTYNLSSTLTLSK

SEQ ID NO: 29 - protein, 8 aa, synthetic construct
  MOD_RES 2: note = Glycosylated
NYNINWVR

SEQ ID NO: 30 - protein, 20 aa, synthetic construct
VDNALQSGNSQENVTEQDSK

SEQ ID NO: 31 - protein, 11 aa, synthetic construct
NQVNSLTCLVK

SEQ ID NO: 32 - protein, 9 aa, synthetic construct
  MOD_RES 5: note = Glycosylated
EEQFNSTYR

SEQ ID NO: 33 - protein, 18 aa, synthetic construct
  MOD_RES 5: note = Glycosylated
EIVLTQSPATLSLSPGER

SEQ ID NO: 34 - protein, 16 aa, synthetic construct
  MOD_RES 7: note = Glycosylated
LLIYDASNRPTGIPAR

SEQ ID NO: 35 - protein, 16 aa, synthetic construct
  MOD_RES 7: note = Glycosylated
LLIYDASNRPTGIPAR

SEQ ID NO: 36 - protein, 18 aa, synthetic construct
  MOD_RES 7: note = Glycosylated
EIVLTQSPATLSLSPGER

SEQ ID NO: 37 - protein, 8 aa, synthetic construct
  MOD_RES 8: note = Glycosylated
VDNALSGN

SEQ ID NO: 38 - protein, 14 aa, synthetic construct
  MOD_RES 11: note = Glycosylated
TYTCNVDHKPNNTK

SEQ ID NO: 39 - protein, 12 aa, synthetic construct
  MOD_RES 2: note = Glycosylated
LNCTASGFTFSR

(19) United States
(12) Patent Application Publication
(10) Pub. No.: US 2025/0067750 A1
(43) Pub. Date: Feb. 27, 2025

(54) AUTOMATION-ENABLED METHODS FOR CHARACTERIZING NON-CONSENSUS N- AND O-GLYCANS IN PROTEINS

(71) Applicant: Regeneron Pharmaceuticals, Inc., Tarrytown, NY (US)

(72) Inventors: Abu Nahiyaan Navid, Long Island City, NY (US); Yunlong Zhao, Scarsdale, NY (US); Yuan Mao, Hartsdale, NY (US); Ning Li, New Canaan, CT (US)

(21) Appl. No.: 18/787,598

(22) Filed: Jul. 29, 2024

Related U.S. Application Data
(60) Provisional application No. 63/529,199, filed on Jul. 27, 2023.

(51) Int. Cl. G01N 33/68; C12Q 1/34; G01N 30/02; G01N 30/72; G01N 30/88; G16B 40/10

(52) U.S. Cl. CPC G01N 33/6848; C12Q 1/34; G01N 30/7233; G01N 33/6854; G16B 40/10; G01N 2030/027; G01N 2030/8831; G01N 2333/98

(57) ABSTRACT

The present invention generally pertains to methods of characterizing non-consensus glycosylation sites of a protein. In particular, the present invention pertains to the use of high-throughput automated processes through digestion, enrichment of glycopeptides by liquid chromatography-mass spectrometry for identifying identification of non-consensus glycosylation sites.

### CROSS-REFERENCE TO RELATED APPLICATIONS

This application claims the benefit of priority to U.S. Provisional Application No. 63/529,199, filed Jul. 27, 2023, which is incorporated herein by reference in its entirety.

### REFERNECE TO SEQUENCE LISTING

This application includes a Sequence Listing filed electronically as an XML file named 381206032SEQ, created on Nov. 5, 2024, with a size of 48,011 bytes. The Sequence Listing is incorporated herein by reference.

### BACKGROUND

Characterization of therapeutic proteins' critical quality attributes (CQAs) is important due to the large size and complex heterogeneity of this major class of therapeutics. One such CQA is any variation in the glycosylation of the protein, which may affect downstream pathways, efficacy, binding capability, and immunogenicity. For example, a fragment crystallizable (Fc) glycosylation at N297 can be required for initiating important immune responses.

Characterizing glycosylation of proteins is critical as the presence of glycosylation can influence binding capability and downstream signaling pathways. A typical consensus glycosylation, for example an N-glycosylation, can occur at any site that follows the motif N*XS/T, where X≠P. Any N-glycosylation that does not follow this motif is considered to be non-consensus. Understanding non-consensus glycosylation presents a particular challenge as they are generally a low abundant species, do not follow the typical motif for glycosylation and can be unpredictable. Characterization of non-consensus glycosylation can lead to a better understanding as to how non-consensus glycosylation may affect safety concerns such as immunogenicity and diminished binding capabilities. Current methods of glycosylation identification are insufficient due to a lack of high-throughput identification methods, as well as inadequate sensitivity.

Therefore, a demand exists for methods to identify non-consensus glycosylation in protein products, and specifically antibody products, in a sensitive, high-throughput manner.

### SUMMARY

A method has been developed for characterizing intact non-consensus glycosylation sites of a protein in a high-throughput process. In an exemplary embodiment, a sample including a protein can be contacted to a digestive enzyme, for example trypsin or a variant thereof, to produce peptides. In one aspect, the digestion may be automated. The peptides may then be subjected to liquid chromatography, which can be hydrophilic interaction chromatography (HILIC), for glycopeptide enrichment. In one aspect, the liquid chromatography step may be automated. The glycopeptides may then be subjected to liquid chromatography-tandem mass spectrometry (LC-MS/MS) for further identification and characterization of glycosylation. The most hydrophilic (last eluted in HILIC) non-glycopeptide for IgG1 and IgG4 is a peptide from the kappa CL domain referred to herein as the “VDNAL peptide” (SEQ ID NO: 1). The VDNAL peptide can therefore be used as a dividing peak between non-glycosylated and glycosylated peptides when separated in order of hydrophilicity or hydrophobicity.

This disclosure provides methods for characterizing intact non-consensus glycosylation sites of a protein in a high-throughput process. In some exemplary embodiments, the methods can comprise: (a) treating said protein with a digestive enzyme to form glycopeptides; (b) subjecting said glycopeptides to hydrophilic interaction liquid chromatography (HILIC) to form enriched glycopeptides; and (c) subjecting said enriched glycopeptides to liquid chromatography-tandem mass spectrometry (LC-MS/MS) analysis by adjusting the Automatic Gain Control (AGC) target and/or maximum injection time to characterize intact non-consensus glycosylation sites of said protein.

In one aspect, said protein is selected from a group consisting of an antibody, a monoclonal antibody, a bispecific antibody, an antibody fragment, an antibody-derived protein, an antigen-binding protein, an antibody-drug conjugate, or a fusion protein.

In one aspect, said protein is denatured and reduced prior to the digestion of step (a).

In one aspect, said digestive enzyme is selected from a group consisting of pepsin, trypsin, Tryp-N, chymotrypsin, Lys-N, Lys-C, Asp-N, Arg-C, Glu-C, papain, IdeS, and variants thereof.

In one aspect, said treating said protein with a digestive enzyme is automated.

In one aspect, said liquid chromatography is automated.

In one aspect, said liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof.

In one aspect, said mass spectrometer is an electrospray ionization mass spectrometer, nano-electrospray ionization mass spectrometer, or an Orbitrap-based mass spectrometer, wherein said mass spectrometer is coupled to said liquid chromatography system.

In one aspect, said AGC target is about 4×10⁴. In another aspect, said AGC target is about 400% of a conventional AGC target.

In one aspect, said maximum injection time is about 250 ms. In one aspect, said maximum injection time is about 400% of a conventional maximum injection time.

In one aspect, said HILIC step further comprises comparing said glycopeptides to a VDNAL peptide peak as a divider between non-glycosylated and glycosylated peptides as related to retention time.

In one aspect, non-consensus glycosylations are selected from the group consisting of N-glycosylation, O-glycosylation and sequence variants of S->N mutations.

In one aspect, said non-consensus glycosylations are identified through comparing the sample to a database of known non-consensus glycosylations. In a specific aspect, said database is the Byonic database.

In one aspect, said protein is treated with an endoglycosidase enzyme prior to the digestion of step (a).

In one aspect, said endoglycosidase enzyme is PNGase F, endoglycosidase F1, endoglycosidase F2, endoglycosidase F3, endoglycosidase H, O-glycosidase, Endo-B-Galactosidase or combinations thereof.

In one aspect, said endoglycosidase enzyme is PNGase F.

This disclosure additionally provides methods for characterizing glycosylation sites of a protein in a high-throughput process. In some exemplary embodiments, the methods can comprise: (a) treating said protein with a digestive enzyme to form glycopeptides; (b) subjecting said glycopeptides to a first liquid chromatography step to form enriched glycopeptides; and (c) subjecting said enriched glycopeptides to a second liquid chromatography step coupled to tandem mass spectrometry (LC-MS/MS) analysis by adjusting the AGC target/time to characterize glycosylation sites of said protein.

In one aspect, said protein is selected from a group consisting of an antibody, a monoclonal antibody, a bispecific antibody, an antibody fragment, an antibody-derived protein, an antigen-binding protein, an antibody-drug conjugate, or a fusion protein.

In one aspect, said protein is denatured and reduced prior to the digestion of step (a).

In one aspect, said digestive enzyme is selected from a group consisting of pepsin, trypsin, Tryp-N, chymotrypsin, Lys-N, Lys-C, Asp-N, Arg-C, Glu-C, papain, IdeS, and variants thereof.

In one aspect, said treating said protein with a digestive enzyme is automated.

In one aspect, said first liquid chromatography step and/or said second liquid chromatography step comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof.

In one aspect, said first liquid chromatography step is hydrophilic interaction liquid chromatography (HILIC).

In one aspect, said first liquid chromatography step and/or said second liquid chromatography step is automated.

In one aspect, said mass spectrometer is an electrospray ionization mass spectrometer, nano-electrospray ionization mass spectrometer, or an Orbitrap-based mass spectrometer, wherein said mass spectrometer is coupled to said liquid chromatography system.

In one aspect, said first liquid chromatography step further comprises comparing said glycopeptides to a VDNAL peptide peak as a divider between non-glycosylated and glycosylated peptides as related to retention time.

In one aspect, glycosylations are identified through comparing the sample to a database of known glycosylations. In a specific aspect, said database is the Byonic database.

In one aspect, said glycosylations are identified as non-consensus glycosylations. In a specific aspect, said non-consensus glycosylations are selected from the group consisting of N-glycosylation, O-glycosylation and sequence variants of S->N mutations.

In one aspect, said protein is treated with an endoglycosidase enzyme prior to the digestion of step (a).

In one aspect, said endoglycosidase enzyme is PNGase F, endoglycosidase F1, endoglycosidase F2, endoglycosidase F3, endoglycosidase H, O-glycosidase, Endo-B-Galactosidase or combinations thereof.

In one aspect, said endoglycosidase enzyme is PNGase F.

In some exemplary embodiments, the methods can comprise: (a) treating said protein with a digestive enzyme to form a mixture of glycopeptides and non-glycopeptides; (b) subjecting said mixture of glycopeptides and non-glycopeptides to hydrophilic interaction liquid chromatography (HILIC) to form enriched glycopeptides; and (c) subjecting said enriched glycopeptides to liquid chromatography-tandem mass spectrometry (LC-MS/MS) analysis by adjusting the Automatic Gain Control (AGC) target and/or maximum injection time to characterize intact non-consensus glycosylation sites of said protein.

In one aspect, said protein is selected from a group consisting of an antibody, a monoclonal antibody, a bispecific antibody, an antibody fragment, an antibody-derived protein, an antigen-binding protein, an antibody-drug conjugate, or a fusion protein.

In one aspect, said protein is denatured and reduced prior to the digestion of step (a).

In one aspect, said digestive enzyme is selected from a group consisting of pepsin, trypsin, Tryp-N, chymotrypsin, Lys-N, Lys-C, Asp-N, Arg-C, Glu-C, papain, IdeS, and variants thereof.

In one aspect, said treating said protein with a digestive enzyme is automated.

In one aspect, said liquid chromatography is automated.

In one aspect, said liquid chromatography comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof.

In one aspect, said mass spectrometer is an electrospray ionization mass spectrometer, nano-electrospray ionization mass spectrometer, or an Orbitrap-based mass spectrometer, wherein said mass spectrometer is coupled to said liquid chromatography system.

In one aspect, said AGC target is about 4×10⁴. In another aspect, said AGC target is about 400% of a conventional AGC target.

In one aspect, said maximum injection time is about 250 ms. In another aspect, said maximum injection time is about 400% of a conventional maximum injection time.

In one aspect, said HILIC step further comprises comparing said glycopeptides to a VDNAL peptide peak as a divider between non-glycosylated and glycosylated peptides as related to retention time.

In one aspect, non-consensus glycosylations are selected from the group consisting of N-glycosylation, O-glycosylation and sequence variants of S->N mutations.

In one aspect, said non-consensus glycosylations are identified through comparing the sample to a database of known non-consensus glycosylations. In a specific aspect, said database is the Byonic database.

In one aspect, said protein is treated with an endoglycosidase enzyme prior to the digestion of step (a).

In one aspect, said endoglycosidase enzyme is PNGase F, endoglycosidase F1, endoglycosidase F2, endoglycosidase F3, endoglycosidase H, O-glycosidase, Endo-B-Galactosidase or combinations thereof.

In one aspect, said endoglycosidase enzyme is PNGase F.

In some exemplary embodiments, the methods can comprise: (a) treating said protein with a digestive enzyme to form a mixture of glycopeptides and non-glycopeptides; (b) subjecting said mixture of glycopeptides and non-glycopeptides to a first liquid chromatography step to form enriched glycopeptides; and (c) subjecting said enriched glycopeptides to a second liquid chromatography step coupled to tandem mass spectrometry (LC-MS/MS) analysis by adjusting the AGC target/time to characterize glycosylation sites of said protein.

In one aspect, said protein is selected from a group consisting of an antibody, a monoclonal antibody, a bispecific antibody, an antibody fragment, an antibody-derived protein, an antigen-binding protein, an antibody-drug conjugate, or a fusion protein.

In one aspect, said protein is denatured and reduced prior to the digestion of step (a).

In one aspect, said digestive enzyme is selected from a group consisting of pepsin, trypsin, Tryp-N, chymotrypsin, Lys-N, Lys-C, Asp-N, Arg-C, Glu-C, papain, IdeS, and variants thereof.

In one aspect, said treating said protein with a digestive enzyme is automated.

In one aspect, said first liquid chromatography step and/or said second liquid chromatography step comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction chromatography, mixed-mode chromatography, or a combination thereof.

In one aspect, said first liquid chromatography step is hydrophilic interaction liquid chromatography (HILIC).

In one aspect, said first liquid chromatography step and/or said second liquid chromatography step is automated.

In one aspect, said mass spectrometer is an electrospray ionization mass spectrometer, nano-electrospray ionization mass spectrometer, or an Orbitrap-based mass spectrometer, wherein said mass spectrometer is coupled to said liquid chromatography system.

In one aspect, said first liquid chromatography step further comprises comparing said glycopeptides to a VDNAL peptide peak as a divider between non-glycosylated and glycosylated peptides as related to retention time.

In one aspect, glycosylations are identified through comparing the sample to a database of known glycosylations. In a specific aspect, said database is the Byonic database.

In one aspect, said glycosylations are identified as non-consensus glycosylations. In a specific aspect, said non-consensus glycosylations are selected from the group consisting of N-glycosylation, O-glycosylation and sequence variants of S->N mutations.

In one aspect, said protein is treated with an endoglycosidase enzyme prior to the digestion of step (a).

In one aspect, said endoglycosidase enzyme is PNGase F, endoglycosidase F1, endoglycosidase F2, endoglycosidase F3, endoglycosidase H, O-glycosidase, Endo-B-Galactosidase or combinations thereof.

In one aspect, said endoglycosidase enzyme is PNGase F.

These, and other aspects of the invention will be better appreciated and understood when considered in conjunction with the following description and accompanying drawings. The following description, while indicating various embodiments and numerous specific details thereof, is given way of illustration and not of limitation. Many substitutions, modifications, additions, or rearrangements may be made within the scope of the invention.

### BRIEF DESCRIPTION OF THE DRAWINGS

This patent or application contains at least one drawing executed in color. Copies of this patent or patent application publication with color drawings will be provided by the Office upon request and payment of the necessary fee.

FIG. 1 illustrates a workflow of an automated glycopeptide enrichment wherein the protein is digested, enriched on a HILIC column, then characterized on LC-MS, followed by data analysis, according to an exemplary embodiment.

FIG. 2 illustrates a workflow for preparing proteins for digestion and data analysis, according to an exemplary embodiment.

FIG. 3A shows IP-HILIC-MS spectra for the delineation of the most hydrophilic non-glycosylated peptide (VDNAL) (SEQ ID NO: 2) and the glycosylated peptides (SEQ ID NO: 3), according to an exemplary embodiment.

FIG. 3B shows IP-HILIC-MS spectra for the delineation of the most hydrophilic non-glycosylated peptide (VDNAL) and the glycosylated peptides from human plasma, according to an exemplary embodiment.

FIG. 4 shows the landmark VDNAL peptide and Fc region glycopeptides. To ensure retention of potential co-eluting low abundant glycopeptide species, collection of fractions commences during elution of the VDNAL peptide.

FIG. 5A shows the enrichment gradient and collection of all glycoproteins over a 10-minute time span, according to an exemplary embodiment.

FIG. 5B shows co-elution of glycopeptides with the non-glycosylated VDNAL sequence, according to an exemplary embodiment.

FIG. 6 illustrates a workflow for the comparison of before and after enrichment of mAb post-digestion, according to an exemplary embodiment. Several serial dilutions of the digested mAb were performed, followed by spiked-in glycoproteins. Half of the samples were enriched and desalted before LC-MS analysis and the other half were only desalted before LC-MS analysis.

FIG. 7 shows several glycopeptides including a previously unreported glycopeptide (highlighted in yellow; i.e. peptide HT*FSGVASVESSSGEAFHVGK, (SEQ ID NO: 4)), according to an exemplary embodiment. All predicted glycopeptides from standard proteins were observed, and relative levels are maintained through glycopeptide enrichment.

FIG. 8A and FIG. 8B show a comparison of enriched and unenriched glycopeptides, according to an exemplary embodiment. Each point in the logarithmic graphs represents the total area of a glycopeptide species/total area of FC glycopeptide against the total MS2 score generated in Byonic. FIG. 8A shows the data for the enriched glycopeptides. FIG. 8B shows the data for the unenriched glycopeptides.

FIG. 9 shows the relative levels of several glycopeptides and their identification scores with and without enrichment, according to an exemplary embodiment.

FIG. 10 shows the relative levels of a glycopeptide with and without enrichment, wherein a small glycan (HexNAc) was not identified after enrichment due to the hydrophobicity of the peptide being closer to that of non-glycosylated peptides, according to an exemplary embodiment.

FIG. 11 shows a validation of glycopeptide enrichment, according to an exemplary embodiment.

FIG. 12 shows MS spectra for enriched versus non-enriched samples, according to an exemplary embodiment. The total ion current chromatogram (TIC) enriched spectrum has a cleaner profile as compared to the non-enriched TIC spectrum. The enriched sample has a higher relative abundance of oxonium ions.

FIG. 13 shows a workflow of filtering data on Byonic commencing with the individual searches through filtering and validation of the data, according to an exemplary embodiment.

FIG. 14A and FIG. 14B show mass spectra of two types of modifications observed in a common region across mAbs, according to an exemplary embodiment. FIG. 14A is for a first mAb and FIG. 14B is for a second mAb.

FIG. 15 shows mass spectra of multiple glycoforms for a single mAb, according to an exemplary embodiment.

FIG. 16 shows glycoforms identified after digestion and LC-MS analysis, according to an exemplary embodiment.

FIG. 17 shows LC-MS spectra of non-human glycoforms on conventional Fc glycosylation sites, according to an exemplary embodiment. The two non-human glycoforms identified are the NeuGc fragments shown in the zoomed in spectrum of the fingerprint region.

FIG. 18 shows the digestion and enrichment for eight replicates of three peptide fragments, according to an exemplary embodiment.

FIG. 19A and FIG. 19B show the results of the digestion for eight replicates of common glycopeptide sequences that were normalized to a common peptide sequence, according to an exemplary embodiment. FIG. 19A compares common glycopeptides to the VDNAL, non-glycosylated peptide and FIG. 19B compares common glycopeptides to the Fc glycopeptide.

FIG. 20 shows non-consensus N-glycosylation sites containing N-glycan motifs due to S->N sequence variants, according to an exemplary embodiment.

FIG. 21A and FIG. 21B show extracted ion chromatograms (XIC) of the top four most abundant glycopeptides from the canonical glycosylation site in Fc region (N297) after HILIC enrichment after limited deglycosylation (FIG. 21A) compared with untreated control (FIG. 21B), according to an exemplary embodiment.

FIGS. 22A&B show MS1 XIC scans (A) and MS2 spectra (B) for a glycopeptide from non-canonical glycosylation site detected after HILIC enrichment with (top panels) and without (bottom panels) limited deglycosylation.

FIGS. 23A&B show MS1 scans and MS2 spectra for an N-glycopeptide from a non-consensus glycosylation site with limited deglycosylation (A) and without limited deglycosylation (B).

### DETAILED DESCRIPTION

Characterization of therapeutic antibodies' critical quality attributes (CQAs) is important due to the large size and complex heterogeneity of this major class of therapeutics. One such CQA is variation in the glycosylation of the protein. Human IgG subclasses share a conserved amino acid sequence in the fragment crystallizable (Fc) region, including a single N-glycosylation site (N297). Fc glycosylation is crucial to the interaction between Fc and different types of receptors, and therefore related to the biophysical profile of an IgG molecule, such as lifetime and effector functions. For instance, antibodies with Fc-afucosylation exhibit stronger binding affinity to Fcγ receptor III and enhanced antibody-dependent cellular cytotoxicity (ADCC); Fc-galactosylation is associated with a strong binding affinity to C1q and enhanced complement-dependent cytotoxicity (CDC); and antibodies with N-linked mannose-5 glycan (Man5) may display higher immunogenicity and faster clearance in serum. Therefore, characterizing the glycan profile of a potential therapeutic protein, such as Fc glycosylation of a therapeutic antibody, is an important aspect of drug development, and methods are needed for effectively doing so in complex samples. The ability to automate characterization will greatly enhance the identification of non-consensus glycosylation. Additionally, high-throughput identification methods, which are disclosed herein, are useful for characterizing potential therapeutic proteins on a timeline compatible with drug development.

Detecting glycosylations on proteins is critical as their presence can influence binding capability and downstream signaling pathways. A typical consensus glycosylation, for example an N-glycosylation, occurs at any site that follows the motif N*XS/T, where X≠P. Any glycosylation that does not follow this motif is considered to be non-consensus. Understanding non-consensus glycosylation has presented a particular challenge, as non-consensus glycosylations are generally a low abundant species, they do not follow the typical motif for glycosylation and are unpredictable. Characterization of non-consensus glycosylation could help to better understand how non-consensus glycosylations may affect safety and efficacy concerns such as immunogenicity and diminished binding capabilities.

Electrospray ionization mass spectrometry (ESI MS)-based intact protein analysis has become an essential tool for the characterization of therapeutic proteins during development. Most commonly, MS is coupled with reversed phase liquid chromatography (RPLC) under denaturing conditions. However, the sensitivity of this method, and the signal-to-noise ratio produced by the resulting complex sample with a wide range of analyte charge states, has limits which may make it unreliable for accurate quantitation of low-abundance antibodies.

As described above, there exists a need for sensitive methods to characterize and identify intact non-consensus glycosylation of proteins and peptides, such as therapeutic antibodies, in a sample. This disclosure sets forth novel HILIC and LC-MS/MS methods for characterizing and identifying glycosylation of proteins and peptides, suitable for development of therapeutic antibodies. In exemplary embodiments, using digestion, HILIC for enrichment, LC-MS/MS methods, and comparison to a landmark hydrophilic non-glycosylated peptide, VDNAL, novel methods were developed for successful identification and characterization of non-consensus glycosylation sites on a therapeutic antibody.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described.

The term “a” should be understood to mean “at least one” and the terms “about” and “approximately” should be understood to permit standard variation as would be understood by those of ordinary skill in the art, and where ranges are provided, endpoints are included. As used herein, the terms “include,” “includes,” and “including” are meant to be non-limiting and are understood to mean “comprise,” “comprises,” and “comprising” respectively.

As used herein, the term “protein” or “protein of interest” can include any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds. “Synthetic peptide or polypeptide” refers to a non-naturally occurring peptide or polypeptide. Synthetic peptides or polypeptides can be synthesized, for example, using an automated polypeptide synthesizer. Various solid phase peptide synthesis methods are known to those of skill in the art. A protein may comprise one or multiple polypeptides to form a single functioning biomolecule. In another exemplary aspect, a protein can include antibody fragments, nanobodies, recombinant antibody chimeras, cytokines, chemokines, peptide hormones, and the like. Proteins of interest can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. Proteins may be produced using recombinant cell-based production systems, such as the insect bacculovirus system, yeast systems (e.g., Pichia sp.), and mammalian systems (e.g., CHO cells and CHO derivatives like CHO-K1 cells). For a recent review discussing biotherapeutic proteins and their production, see Ghaderi et al., “Production platforms for biotherapeutic glycoproteins. Occurrence, impact, and challenges of non-human sialylation” (Darius Ghaderi et al., Production platforms for biotherapeutic glycoproteins. Occurrence, impact, and challenges of non-human sialylation, 28 BIOTECHNOLOGY AND GENETIC ENGINEERING REVIEWS 147-176 (2012), the entire teachings of which are herein incorporated). In some exemplary embodiments, proteins comprise modifications, adducts, and other covalently linked moieties. These modifications, adducts and moieties include, for example, avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein of interest can be a recombinant protein, an in vivo product of gene therapy, a therapeutic protein, an antibody, a bispecific antibody, a multispecific antibody, antibody fragment, monoclonal antibody, antigen-binding protein, fusion protein, scFv, a multisubunit protein, a receptor, a receptor ligand, and combinations thereof.

As used herein, the term “recombinant protein” refers to a protein produced as the result of the transcription and translation of a gene carried on a recombinant expression vector that has been introduced into a suitable host cell. In certain exemplary embodiments, the recombinant protein can be an antibody, for example, a chimeric, humanized, or fully human antibody. In certain exemplary embodiments, the recombinant protein can be an antibody of an isotype selected from group consisting of: IgG, IgM, IgA1, IgA2, IgD, or IgE. In certain exemplary embodiments the antibody molecule is a full-length antibody (e.g., an IgG1) or alternatively the antibody can be a fragment (e.g., an Fc fragment or a Fab fragment).

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4. An amino acid consensus sequence may be defined based on a side-by-side analysis of two or more CDRs. The term “antibody,” as used herein, also includes antigen-binding fragments of full antibody molecules.

The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab' fragment, a F(ab′)2 fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. In some exemplary embodiments, an antibody fragment comprises a sufficient amino acid sequence of the parent antibody of which it is a fragment that it binds to the same antigen as does the parent antibody; in some exemplary embodiments, a fragment binds to the antigen with a comparable affinity to that of the parent antibody and/or competes with the parent antibody for binding to the antigen. An antibody fragment may be produced by any means. For example, an antibody fragment may be enzymatically or chemically produced by fragmentation of an intact antibody and/or it may be recombinantly produced from a gene encoding the partial antibody sequence. Alternatively, or additionally, an antibody fragment may be wholly or partially synthetically produced. An antibody fragment may optionally comprise a single chain antibody fragment. Alternatively, or additionally, an antibody fragment may comprise multiple chains that are linked together, for example, by disulfide linkages. An antibody fragment may optionally comprise a multi-molecular complex. A functional antibody fragment typically comprises at least about 50 amino acids and more typically comprises at least about 200 amino acids.

The term “bispecific antibody” includes an antibody capable of selectively binding two or more epitopes. Bispecific antibodies generally comprise two different heavy chains with each heavy chain specifically binding a different epitope—either on two different molecules (e.g., antigens) or on the same molecule (e.g., on the same antigen). If a bispecific antibody is capable of selectively binding two different epitopes (a first epitope and a second epitope), the affinity of the first heavy chain for the first epitope will generally be at least one to two or three or four orders of magnitude lower than the affinity of the first heavy chain for the second epitope, and vice versa. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions and such sequences can be expressed in a cell that expresses an immunoglobulin light chain.

A typical bispecific antibody has two heavy chains each having three heavy chain CDRs, followed by a CH1 domain, a hinge, a CH2 domain, and a CH3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes. BsAbs can be divided into two major classes, those bearing an Fc region (IgG-like) and those lacking an Fc region, the latter normally being smaller than the IgG and IgG-like bispecific molecules comprising an Fc. The IgG-like bsAbs can have different formats such as, but not limited to, triomab, knobs into holes IgG (kih IgG), crossMab, orth-Fab IgG, Dual-variable domains Ig (DVD-Ig), two-in-one or dual action Fab (DAF), IgG-single-chain Fv (IgG-scFv), or κλ-bodies. The non-IgG-like different formats include tandem scFvs, diabody format, single-chain diabody, tandem diabodies (TandAbs), Dual-affinity retargeting molecule (DART), DART-Fc, nanobodies, or antibodies produced by the dock-and-lock (DNL) method (Gaowei Fan, Zujian Wang & Mingju Hao, Bispecific antibodies and their applications, 8 JOURNAL OF HEMATOLOGY & ONCOLOGY 130; Dafne Müller & Roland E. Kontermann, Bispecific Antibodies, HANDBOOK OF THERAPEUTIC ANTIBODIES 265-310 (2014), the entire teachings of which are herein incorporated). The methods of producing bsAbs are not limited to quadroma technology based on the somatic fusion of two different hybridoma cell lines, chemical conjugation, which involves chemical cross-linkers, and genetic approaches utilizing recombinant DNA technology. Examples of bsAbs include those disclosed in the following patent applications, which are hereby incorporated by reference: U.S. Ser. No. 12/823,838, filed Jun. 25, 2010; U.S. Ser. No. 13/488,628, filed Jun. 5, 2012; U.S. Ser. No. 14/031,075, filed Sep. 19, 2013; U.S. Ser. No. 14/808,171, filed Jul. 24, 2015; U.S. Ser. No. 15/713,574, filed Sep. 22, 2017; U.S. Ser. No. 15/713,569, field Sep. 22, 2017; U.S. Ser. No. 15/386,453, filed Dec. 21, 2016; U.S. Ser. No. 15/386,443, filed Dec. 21, 2016; U.S. Ser. No. 15/22343 filed Jul. 29, 2016; and U.S. Ser. No. 15/814,095, filed Nov. 15, 2017.

As used herein, “multispecific antibody” refers to an antibody with binding specificities for at least two different antigens. While such molecules normally will only bind two antigens (i.e., bispecific antibodies, bsAbs), antibodies with additional specificities such as trispecific antibody and KIH Trispecific can also be addressed by the system and method disclosed herein.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, phage display technologies, gene therapy, or a combination thereof.

In some exemplary embodiments, the protein of interest can be produced from mammalian cells. The mammalian cells can be of human origin or non-human origin can include primary epithelial cells (e.g., keratinocytes, cervical epithelial cells, bronchial epithelial cells, tracheal epithelial cells, kidney epithelial cells and retinal epithelial cells), established cell lines and their strains (e.g., HEK293 embryonic kidney cells, BHK cells, HeLa cervical epithelial cells and PER-C6 retinal cells, MDBK (NBL-1) cells, 911 cells, CRFK cells, MDCK cells, CHO cells, BeWo cells, Chang cells, Detroit 562 cells, HeLa 229 cells, HeLa S3 cells, Hep-2 cells, KB cells, LSI80 cells, LS174T cells, NCI-H-548 cells, RPMI2650 cells, SW-13 cells, T24 cells, WI-28 VA13, 2RA cells, WISH cells, BS-C-I cells, LLC-MK2 cells, Clone M-3 cells, 1-10 cells, RAG cells, TCMK-1 cells, Y-1 cells, LLC-PKi cells, PK(15) cells, GHi cells, GH3 cells, L2 cells, LLC-RC 256 cells, MHiCi cells, XC cells, MDOK cells, VSW cells, and TH-I, B1 cells, BSC-1 cells, RAf cells, RK-cells, PK-15 cells or derivatives thereof), fibroblast cells from any tissue or organ (including but not limited to heart, liver, kidney, colon, intestines, esophagus, stomach, neural tissue (brain, spinal cord), lung, vascular tissue (artery, vein, capillary), lymphoid tissue (lymph gland, adenoid, tonsil, bone marrow, and blood), spleen, and fibroblast and fibroblast-like cell lines (e.g., CHO cells, TRG-2 cells, IMR-33 cells, Don cells, GHK-21 cells, citrullinemia cells, Dempsey cells, Detroit 551 cells, Detroit 510 cells, Detroit 525 cells, Detroit 529 cells, Detroit 532 cells, Detroit 539 cells, Detroit 548 cells, Detroit 573 cells, HEL 299 cells, IMR-90 cells, MRC-5 cells, WI-38 cells, WI-26 cells, Midi cells, CHO cells, CV-1 cells, COS-1 cells, COS-3 cells, COS-7 cells, Vero cells, DBS-FrhL-2 cells, BALB/3T3 cells, F9 cells, SV-T2 cells, M-MSV-BALB/3T3 cells, K-BALB cells, BLO-11 cells, NOR-10 cells, C3H/IOTI/2 cells, HSDMiC3 cells, KLN205 cells, McCoy cells, Mouse L cells, Strain 2071 (Mouse L) cells, L-M strain (Mouse L) cells, L-MTK′ (Mouse L) cells, NCTC clones 2472 and 2555, SCC-PSA1 cells, Swiss/3T3 cells, Indian muntjac cells, SIRC cells, Cn cells, and Jensen cells, Sp2/0, NSO, NS1 cells or derivatives thereof).

In some exemplary embodiments, the sample including the protein of interest can be prepared prior to or following enrichment steps, separation steps, and/or analysis steps. Preparation steps can include alkylation, reduction, denaturation, and/or digestion.

As used herein, the term “protein alkylating agent” refers to an agent used for alkylating certain free amino acid residues in a protein. Non-limiting examples of protein alkylating agents are iodoacetamide (IOA), chloroacetamide (CAA), acrylamide (AA), N-ethylmaleimide (NEM), methyl methanethiosulfonate (MMTS), and 4-vinylpyridine or combinations thereof.

As used herein, “protein denaturing” can refer to a process in which the three-dimensional shape of a molecule is changed from its native state. Protein denaturation can be carried out using a protein denaturing agent. Non-limiting examples of a protein denaturing agent include heat, high or low pH, reducing agents like DTT (see below) or exposure to chaotropic agents. Several chaotropic agents can be used as protein denaturing agents. Chaotropic solutes increase the entropy of the system by interfering with intramolecular interactions mediated by non-covalent forces such as hydrogen bonds, van der Waals forces, and hydrophobic effects. Non-limiting examples for chaotropic agents include butanol, ethanol, guanidinium chloride, lithium perchlorate, lithium acetate, magnesium chloride, phenol, propanol, sodium dodecyl sulfate, thiourea, N-lauroylsarcosine, urea, and salts thereof.

As used herein, the term “protein reducing agent” refers to the agent used for reduction of disulfide bridges in a protein. Non-limiting examples of protein reducing agents used to reduce a protein are dithiothreitol (DTT), ß-mercaptoethanol, Ellman's reagent, hydroxylamine hydrochloride, sodium cyanoborohydride, tris(2-carboxyethyl)phosphine hydrochloride (TCEP-HCl), or combinations thereof. A conventional method of protein analysis, reduced peptide mapping, involves protein reduction prior to LC-MS analysis. In contrast, non-reduced peptide mapping omits the sample preparation step of reduction in order to preserve endogenous disulfide bonds. In some exemplary embodiments, non-reduced preparation may be used, for example, in order to preserve an endogenous disulfide bond between Fab arms of an antibody or antibody-derived protein. In other exemplary embodiments, partially-reduced preparation may be used, for example, in order to reduce the disulfide bond between Fab arms of an antibody or antibody-derived protein without fully reducing the protein.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “digestive enzyme” refers to any of a large number of different agents that can perform digestion of a protein. Non-limiting examples of hydrolyzing agents that can carry out enzymatic digestion include protease from Aspergillus Saitoi, elastase, subtilisin, protease XIII, pepsin, trypsin, Tryp-N, chymotrypsin, aspergillopepsin I, LysN protease (Lys-N), LysC endoproteinase (Lys-C), endoproteinase Asp-N (Asp-N), endoproteinase Arg-C (Arg-C), endoproteinase Glu-C (Glu-C) or outer membrane protein T (OmpT), immunoglobulin-degrading enzyme of Streptococcus pyogenes (IdeS), thermolysin, papain, pronase, V8 protease or biologically active fragments or homologs thereof or combinations thereof. For a recent review discussing the available techniques for protein digestion see Switazar et al., “Protein Digestion: An Overview of the Available Techniques and Recent Developments” (Linda Switzar, Martin Giera & Wilfried M. A. Niessen, Protein Digestion: An Overview of the Available Techniques and Recent Developments, 12 JOURNAL OF PROTEOME RESEARCH 1067-1077 (2013)).

In some exemplary embodiments, IdeS or a variant thereof is used to cleave an antibody below the hinge region, producing an Fc fragment and a Fab₂ fragment. Digestion of an analyte may be advantageous because size reduction may increase the sensitivity and specificity of characterization and detection of the analyte using LC-MS. When used for this purpose, digestion that separates out an Fc fragment and keeps a Fab₂ fragment for analysis may be preferred. This is because variable regions of interest, such as the complementarity-determining region (CDR) of an antibody, are contained in the Fab₂ fragment, while the Fc fragment may be relatively uniform between antibodies and thus provide less relevant information. Alternatively, or additionally, digestion that separates out a Fab₂ fragment and keeps an Fc fragment for analysis may be preferred, because the Fc fragment contains an N-glycosylation site of interest.

IdeS digestion has a high efficiency, allowing for high recovery of an analyte. The digestion and elution process may be performed under native conditions, allowing for simple coupling to a native LC-MS system. IdeS or variants thereof are commercially available and may be marketed as, for example, FabRICATOR® or FabRICATOR Z®.

As used herein, a “sample” can be obtained from any step of the bioprocess, such as cell culture fluid (CCF), harvested cell culture fluid (HCCF), any step in the downstream processing, drug substance (DS), or a drug product (DP) comprising the final formulated product. In some other specific exemplary embodiments, the sample can be selected from any step of the downstream process of clarification, chromatographic production, viral inactivation, or filtration. In some specific exemplary embodiments, the drug product can be selected from manufactured drug product in the clinic, shipping, storage, or handling.

In some exemplary embodiments, the sample is a biological sample. As used herein, the term “biological sample” refers to a sample taken from a living organism, for example a human or non-human mammal. A biological sample may comprise or consist of, for example, whole blood, plasma, serum, saliva, tears, semen, cheek tissue, organ tissue, urine, feces, skin, or hair. A sample may be taken from a patient, for example, a clinical sample. In some exemplary embodiments, a sample may be taken from a non-human animal, for example, a preclinical sample. In some exemplary embodiments, a sample may be taken from a non-human animal subjected to gene therapy in order to produce at least one protein of interest that may be included in the sample. In some embodiments, a sample is a further processed form of any of the aforementioned examples of samples.

In some exemplary embodiments, the method for characterizing and/or identifying a glycopeptide, for example an intact N- or O-linked glycopeptide, can optionally comprise enriching a protein of interest in the sample matrix using immunoprecipitation (IP). As used herein, the term “immunoprecipitation” can include a process of precipitating a protein antigen out of solution using an antibody that specifically binds to that particular protein. Immunoprecipitation may be direct, in which antibodies for the target protein are immobilized on a solid-phase substrate, or indirect, in which free antibodies are added to the protein mixture and later captured with, for example, protein A/G beads. In some exemplary embodiments, IP may be conducted under native or near-native conditions, such that the native structure of a protein or proteins of interest are substantially preserved; for example, heavy chain and light chain pairing of an antibody or antibody-derived protein of interest.

In some exemplary embodiments, the solid-phase substrate may be beads, for example agarose beads or magnetic beads. Beads may be coated in streptavidin in order to facilitate adherence to an antibody. A biotinylated “capture” antibody may then be contacted to the streptavidin-coated beads, adhering to the beads and forming “immunoprecipitation beads” capable of binding to the antigen of the adhered antibody. In some exemplary embodiments, the adhered capture antibody may be an anti-Fc antibody, and may specifically be an anti-human Fc antibody. An anti-human Fc antibody will preferentially bind to the Fc domain of any human antibody, and thus may be used to immunoprecipitate or “pull down” a human antibody from a sample, allowing it to be enriched for analysis.

As used herein, the term “liquid chromatography” refers to a process in which a biological/chemical mixture carried by a liquid can be separated into components as a result of differential distribution of the components as they flow through (or into) a stationary liquid or solid phase. Non-limiting examples of liquid chromatography include reversed phase (RP) liquid chromatography, ion-exchange (IEX) chromatography, size exclusion chromatography (SEC), affinity chromatography, hydrophobic interaction chromatography (HIC), hydrophilic interaction chromatography (HILIC), or mixed-mode chromatography (MMC).

The term “hydrophilic interaction chromatography” or HILIC is intended to include a process employing a hydrophilic stationary phase and a hydrophobic organic mobile phase in which hydrophilic compounds are retained longer than hydrophobic compounds. In certain embodiments, the process utilizes a water-miscible solvent mobile phase.

As used herein, the term “fraction analysis” refers to a process in which the components separated by liquid chromatography are collected/captured in vessels, microcentrifuge tubes or 96-well plates. Samples in each fraction may be subjected to additional analyses, such as nano LC-MS/MS.

As used herein, the term “enrichment” refers to a process in which a sample or component(s) of a sample, for example a component separated by liquid chromatography, is concentrated for further analysis and/or identification. Non-limiting examples of enrichment methods include centrifugation, precipitation, electrophoresis and chromatography. An enrichment method can take from 5-15 minutes, about 5 minutes, about 6 minutes, about 7 minutes, about 8 minutes, about 9 minutes, about 10 minutes, about 11 minutes, about 12 minutes, about 13 minutes, about 14 minutes or about 15 minutes per sample. In one aspect, an enrichment method can take about 10 minutes.

As used herein, the term “mass spectrometer” includes a device capable of identifying specific molecular species and measuring their accurate masses. The term is meant to include any molecular detector with which a polypeptide or peptide may be characterized. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization) or through separate processes. The choice of ion source depends on the application.

In some exemplary embodiments, the mass spectrometer can be a tandem mass spectrometer. As used herein, the term “tandem mass spectrometry” includes a technique where structural information on sample molecules is obtained by using multiple stages of mass selection and mass separation. A prerequisite is that the sample molecules be transformed into a gas phase and ionized so that fragments are formed in a predictable and controllable fashion after the first mass selection step. Multistage MS/MS, or MSⁿ, can be performed by first selecting and isolating a precursor ion (MS²), fragmenting it, isolating a primary fragment ion (MS³), fragmenting it, isolating a secondary fragment (MS⁴), and so on, as long as one can obtain meaningful information, or the fragment ion signal is detectable. Tandem MS has been successfully performed with a wide variety of analyzer combinations. Which analyzers to combine for a certain application can be determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time, mass spectrometer ions produced in the ion source can be trapped, isolated, fragmented, and m/z separated in the same physical device. The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization includes, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “mass analyzer” includes a device that can separate species, that is, atoms, molecules, or clusters, according to their mass. Non-limiting examples of mass analyzers that could be employed are time-of-flight (TOF), magnetic electric sector, quadrupole mass filter (Q), quadrupole ion trap (QIT), orbitrap, Fourier transform ion cyclotron resonance (FTICR), and also the technique of accelerator mass spectrometry (AMS).

In some exemplary aspects, the mass spectrometer can work on nanoelectrospray or nanospray. The term “nanoelectrospray” or “nanospray” as used herein refers to electrospray ionization at a very low solvent flow rate, typically hundreds of nanoliters per minute of sample solution or lower, often without the use of an external solvent delivery. The electrospray infusion setup forming a nanoelectrospray can use a static nanoclectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

In some exemplary embodiments, mass spectrometry analysis may use automatic gain control (AGC). AGC may provide automated regulation to a dynamic ion flux transmitted from the source of the instrument, resulting in a more constant ion population in the mass analyzer to adjust for a broad range of relative abundances in a sample.

As used herein, the term “automatic gain control” (AGC) refers to an automated regulation to a dynamic ion flux transmitted from a source to the instrument, common in LC-MS. AGC accomplishes regulation by monitoring ion production from an ion source and provides regulation to adjust and control the number of ions in an ion trap, avoiding saturation and/or space change effects. Conventional methods may use an AGC target of about 1×10⁴, with a resolution of about 15,000 and a maximum injection time of about 60 ms. In some exemplary embodiments, an AGC target may be from about 200% to about 600%, about 200%, about 300%, about 400%, about 500%, or about 600% compared to a conventional AGC target. In some exemplary embodiments, an AGC target may be from about 3×10⁴ to about 5×10⁴, about 3×10⁴, about 4×10⁴, or about 5×10⁴.

In some exemplary embodiments, a maximum injection time may be from about 100 ms to about 500 ms, about 100 ms, about 150 ms, about 200 ms, about 250 ms, about 300 ms, about 350 ms, about 400 ms, about 450 ms, or about 500 ms. In some exemplary embodiments, a resolution may be from about 50,000 to about 70,000, about 50,000, about 60,000, or about 70,000.

In some exemplary embodiments, the mass spectrometer can be coupled to a liquid chromatography-multiple reaction monitoring system. More generally, a mass spectrometer may be capable of analysis by selected reaction monitoring (SRM), including consecutive reaction monitoring (CRM) and parallel reaction monitoring (PRM).

As used herein, “multiple reaction monitoring” or “MRM” refers to a mass spectrometry-based technique that can precisely quantify small molecules, peptides, and proteins within complex matrices with high sensitivity, specificity and a wide dynamic range (Paola Picotti & Ruedi Aebersold, Selected reaction monitoring-based proteomics: workflows, potential, pitfalls and future directions, 9 NATURE METHODS 555-566 (2012)). MRM can be typically performed with triple quadrupole mass spectrometers wherein a precursor ion corresponding to the selected small molecules/peptides is selected in the first quadrupole and a fragment ion of the precursor ion is selected for monitoring in the third quadrupole (Yong Seok Choi et al., Targeted human cerebrospinal fluid proteomics for the validation of multiple Alzheimers disease biomarker candidates, 930 JOURNAL OF CHROMATOGRAPHY B 129-135 (2013)).

In some exemplary embodiments, LC-MS can be performed under native conditions. As used herein, the term “native conditions” can include performing mass spectrometry under conditions that preserve non-covalent interactions in an analyte. Native mass spectrometry is an approach to study intact biomolecular structure in the native or near-native state. The term “native” refers to the biological status of the analyte in solution prior to subjecting to the ionization. Several parameters, such as pH and ionic strength, of the solution containing the biological analytes can be controlled to maintain the native folded state of the biological analytes in solution. Commonly, native mass spectrometry is based on electrospray ionization, wherein the biological analytes are sprayed from a nondenaturing solvent. Other terms, such as noncovalent, native spray, electrospray ionization, nondenaturing, macromolecular, or supramolecular mass spectrometry can also be describing native mass spectrometry. In exemplary embodiments, native MS allows for better spatial resolution compared to non-native MS, improving detection of biotransformation products of a therapeutic protein. For detailed review on native MS, refer to the review: Elisabetta Boeri Erba & Carlo Pe-tosa, The emerging role of native mass spectrometry in characterizing the structure and dynamics of macromolecular complexes, 24 PROTEIN SCIENCE 1176-1192 (2015).

As used herein, the term “consensus” refers to N-glycosylation occurring at any site that follows the motif N*XS/T, where X cannot be a proline (P). As used herein, the term “non-consensus” refers to glycosylations that do not follow the motif N*XS/T.

As used herein, the term “sequence variant” refers to an amino acid substitution that may occur during protein synthesis, replacing one amino acid with another amino acid.

As used herein, the term “hot spot” refers to a common motif or site that may frequently be glycosylated across multiple proteins. In some embodiments, the method of the present invention may be used to identify glycosylation hot spots across proteins of interest, for example therapeutic antibodies. The identification of glycosylation hot spots may be useful for therapeutic protein development.

As used herein, the term “glycan” refers to a chain-like structure consisting of sugar molecules linked together. As used herein, the term “glycosylation” refers to the covalent addition of sugar moieties to specific amino acids on a protein or peptide, forming a glycoprotein or glycopeptide. Non-limiting examples of glycosylations are N-glycosylation, O-glycosylation, C-glycosylation, glypiation and phosphoglycosylation. As used herein, the term “glycoform” is a glycoprotein that differs from one another due to the presence of either different saccharides attached at a specific amino acid or a different type of saccharide attached at a specific amino acid. As used herein, the term “glycosylation analysis” refers to the characterization of glycoproteins. Non-limiting examples of glycosylation analysis that could be employed are characterizing glycan compositions and structures, identifying glycoform heterogeneity and quantifying relative occupancy for each glycoform located on specific glycosylation sites.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools”. Bioinformatics tools provide the capacity to search uninterpreted MS/MS spectra against all possible sequences in the database(s), and provide interpreted (annotated) MS/MS spectra as an output. Non-limiting examples of such tools are Mascot (matrixscience.com), Spectrum Mill (chem.agilent.com), PLGS (waters.com), PEAKS (bioinformaticssolutions.com), Proteinpilot (download.appliedbiosystems.com//proteinpilot), Phenyx (phenyx-ms.com), Sorcerer (sagenresearch.com), OMSSA (pubchem.ncbi.nlm.nih.gov/omssa/), X!Tandem (thegpm.org/TANDEM/), Protein Prospector (prospector.ucsf.edu/prospector/mshome.htm), Byonic (proteinmetrics.com/products/byonic) or Sequest (fields.scripps.edu/sequest).

As used herein, the term “endoglycosidase enzyme” refers to an enzyme that releases oligosaccharides from glycoproteins or glycolipids. It may also cleave polysaccharide chains between residues. Non-limiting examples of endoglycosidase enzymes that can carry out limited deglycosylation include, Peptide: N-glycosidase F (PNGase F), endoglycosidase F1 (Endo F1), endoglycosidase F2 (Endo F2), endoglycosidase F3 (Endo F3), endoglycosidase H (Endo H), O-glycosidase, Endo-B-Galactosidase or biologically active fragments or homologs thereof or combinations thereof.

It is understood that the present invention is not limited to any of the aforesaid protein(s), antibody(s), monoclonal antibody(s), bispecific antibody(s), protein expression system(s), multisubunit protein(s), protein alkylating agent(s), protein denaturing agent(s), protein reducing agent(s), digestive enzyme(s), hydrolyzing agent(s), endoglycosidase enzyme(s), sample(s), solid phase substrate(s), capture antibody(s), liquid chromatography system(s), mobile phase(s), mass spectrometer(s), database(s), or bioinformatics tool(s), and any protein(s), antibody(s), monoclonal antibody(s), bispecific antibody(s), protein expression system(s), multisubunit protein(s), protein alkylating agent(s), protein denaturing agent(s), protein reducing agent(s), digestive enzyme(s), hydrolyzing agent(s), sample(s), solid phase substrate(s), capture antibody(s), liquid chromatography system(s), mobile phase(s), mass spectrometer(s), database(s), or bioinformatics tool(s) can be selected by any suitable means.

The present invention will be more fully understood by reference to the following Examples. They should not, however, be construed as limiting the scope of the invention.

### EXAMPLES

### Example 1. A Liquid Chromatography Workflow for Peptide Characterization

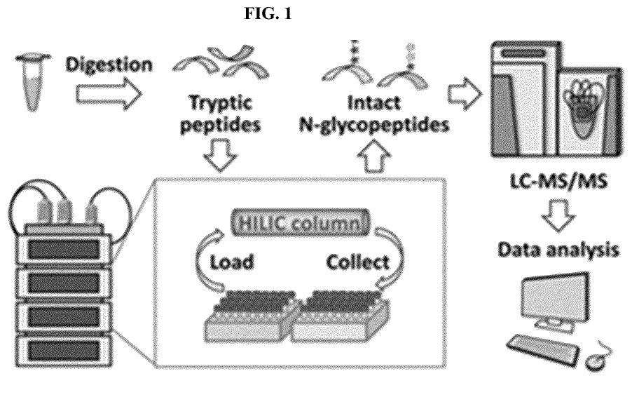

A method for the characterization of non-consensus N- and O-glycans in monoclonal antibodies (mAb) was developed based on hydrophilic interaction chromatography (HILIC) enrichment followed by liquid chromatography-tandem mass spectrometry (LC-MS/MS). Part of an exemplary workflow of the novel method is illustrated in FIG. 1 (Liu et al., 2021, Anal. Chem. 93 (20): 7473-7480). The workflow includes digestion of a protein of interest to form peptides, for example tryptic peptides resulting from trypsin digestion. Peptides may then be loaded onto a HILIC column, allowing for separation of non-glycopeptides from glycopeptides and collection of an enriched glycopeptide sample, optionally using an automated collection approach. Enriched, intact N-glycopeptides collected from the HILIC eluate may then be subjected to LC-MS/MS analysis.

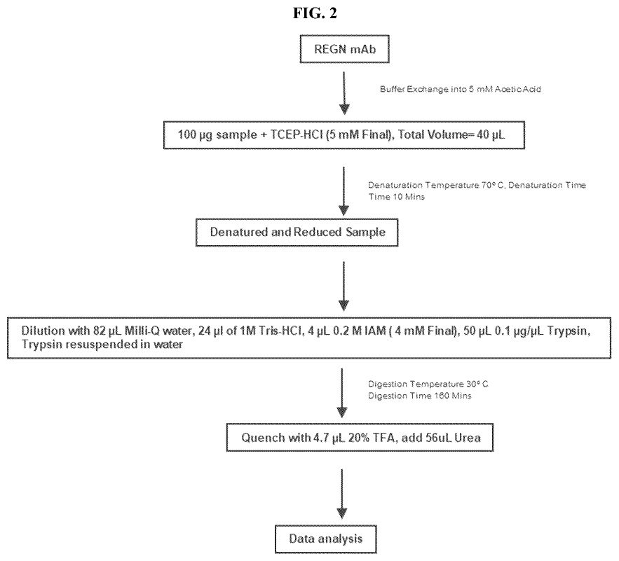

A benefit of the method of the present invention is the compatibility with automation, thus improving the efficiency of the process. An exemplary automated digestion process, for example using a Biomek i5 Automated Workstation, is shown in FIG. 2. Processing steps that can be automated include: buffer exchange, denaturation, reduction, digestion, dilution, and quenching, followed by data analysis.

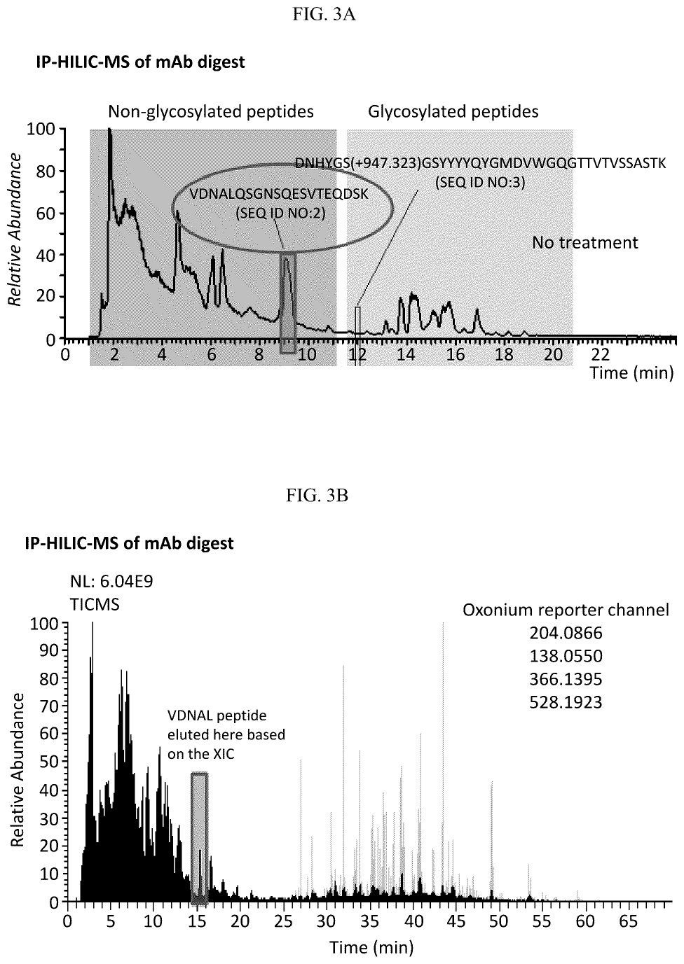

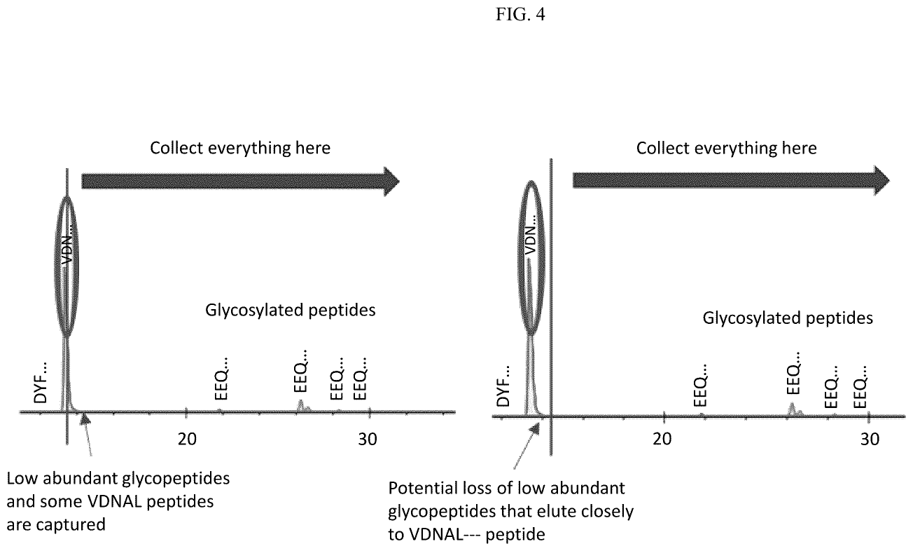

The mobile phase gradient for HILIC separation of glycopeptides from non-glycopeptides was optimized. A mAb was digested, subjected to immunoprecipitation, separated using a HILIC column, and analyzed using mass spectrometry, as shown in FIG. 3A. The most hydrophilic, and therefore last eluted, non-glycopeptide for IgG1 and IgG4 was identified, referred to herein as the “VDNAL peptide”. A similar analysis was undertaken using a sample of human plasma, and in the context of thousands of human plasma glycopeptides, the VDNAL peptide remained the last eluted non-glycopeptide, as shown in FIG. 3B. The extracted ion chromatograms of selected peptides, including the VDNAL peptide and Fc region glycopeptides is shown in FIG. 4. The potential loss of low-abundant glycopeptides may occur if collection started after elution of the VDNAL peptide. To ensure retention of potential co-eluting low abundant glycopeptide species, collection of fractions was commenced during elution of the VDNAL peptide. Therefore, the VDNAL peptide was used as a landmark to delineate glycopeptides from non-glycopeptides in terms of HILIC retention time. Using this landmark, the optimized mobile phase A was 0.045% trifluoroacetic acid (TFA) in water and the optimized mobile phase B was 0.045% TFA in acetonitrile (ACN).

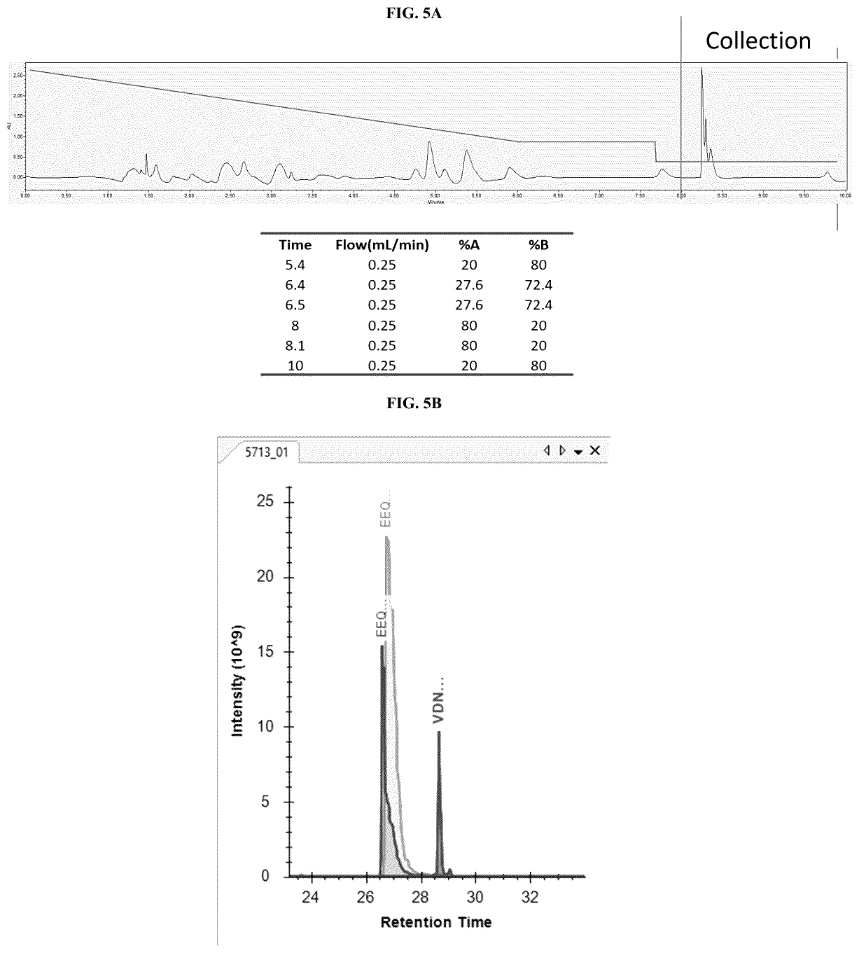

An optimized HILIC gradient and collection strategy allowing for a 10-minute glycopeptide enrichment is shown in FIG. 5A. The percent of mobile phase A and mobile phase B over time is shown. An enriched glycopeptide sample was captured between 8 and 10 minutes. A reversed phase liquid chromatography-mass spectrometry (RPLC-MS) analysis of the enriched glycopeptide sample, illustrating the successful capture of the VDNAL peptide and an exemplary glycopeptide labeled “EEQ”, is shown in FIG. 5B. The mobile phases used for analysis were a mobile phase A of 0.1% formic acid (FA) in water and mobile phase B of 0.1% FA in ACN.

This example demonstrates that a mAb of interest in a sample can be effectively characterized using a method including digestion to form peptide fragments and a novel 10-minute hydrophilic interaction chromatography enrichment using a landmark VDNAL peptide peak for separation of glycopeptides and non-glycopeptides. In particular, using a trypsin digest in combination with a shortened chromatography method allowed for the clear identification of separate glycopeptides.

### Example 2. Validation of the HILIC Enrichment Method Utilizing Spiked-In Standard Glycoproteins

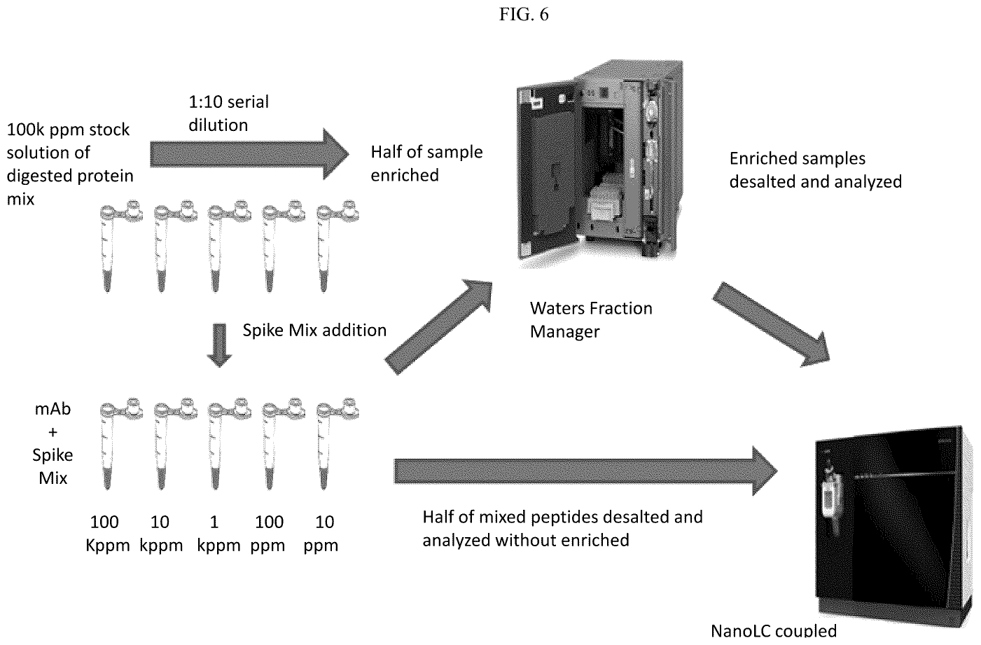

In order to validate the effectiveness and sensitivity of the novel method described in Example 1, the method was tested using standard glycoproteins and an exemplary mAb (mAb1). FIG. 6 shows an exemplary workflow of a process using spiked-in standard glycoproteins to test the enrichment sensitivity of the method of the present invention. A trypsin digest was prepared for mAb1 and for a “spike mix” of other glycoproteins, including bovine fetuin-A/B mix, human serotransferrin, and a therapeutic mini-trap. A stock solution of 100k ppm (relative to the mAb1 concentration) of the digested spike mix was prepared and serially diluted to between 100k ppm and 10 ppm. The various dilutions of the spike mix were added to the digested mAb1. The samples were then divided into equal parts, wherein one part was enriched through the HILIC method described in Example 1 and the other part was not enriched. Both sets of samples were desalted and analyzed by nano LC coupled to an Orbitrap Eclipse MS to evaluate the detection limit of the assay.

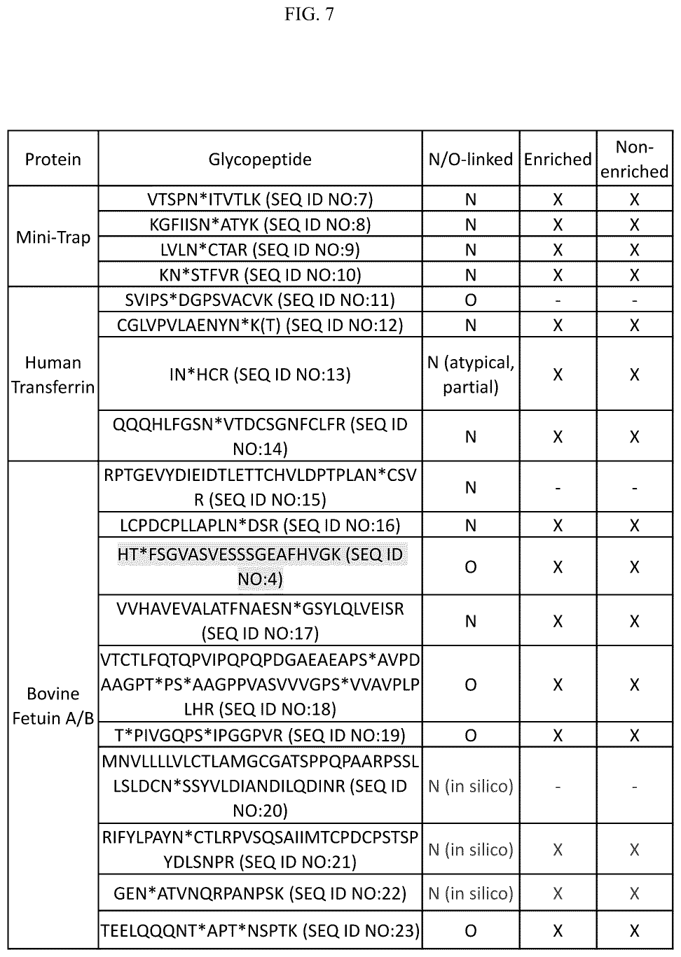

First, glycopeptides from the spike mix glycopeptide standards that were identified using the method of the present invention were compared against a list of glycopeptide species previously reported in the literature, as shown in FIG. 7. Using the method of the present invention, one previously unidentified glycopeptide was identified, which is highlighted in yellow (i.e. peptide HT*FSGVASVESSSGEAFHVGK (SEQ ID NO: 4)). No glycopeptides were lost in the enriched sample compared to the unenriched sample.

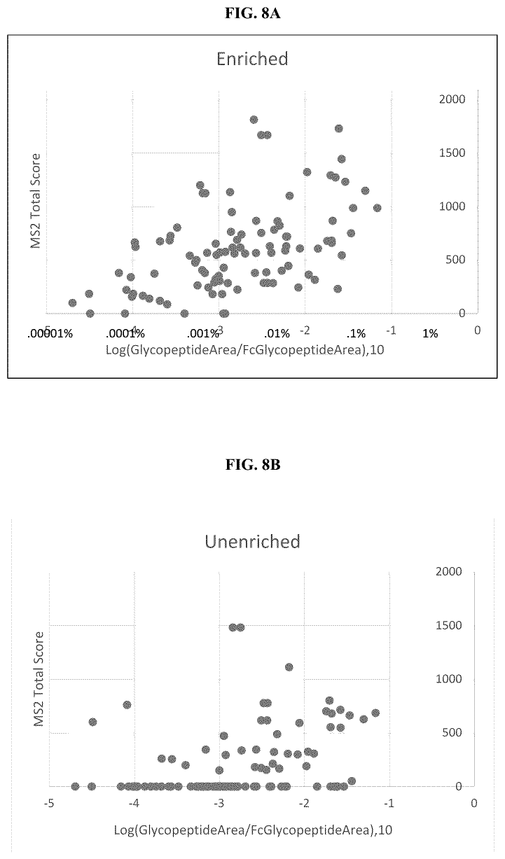

FIGS. 8A&B show a comparison of glycopeptides identified in samples enriched using the method of the present invention compared to unenriched samples. Each point in the logarithmic graphs represents the log of the area under the curve of a glycopeptide species divided by the area under the curve of the Fc glycopeptide. The Fc glycopeptide was chosen for this representation because it is the most frequently occurring glycopeptide, and therefore provides an easily understandable frame of reference for the relative abundance of other glycopeptides. The calculated value was plotted against the total MS2 score generated in Byonic. FIG. 8A shows the data for the enriched glycopeptides. FIG. 8B shows the data for the unenriched glycopeptides. A clear trend is apparent where glycopeptides detected at lower levels also have a lower MS2 score. The enriched fraction had a significant improvement in the MS2 scoring compared to the unenriched fraction.

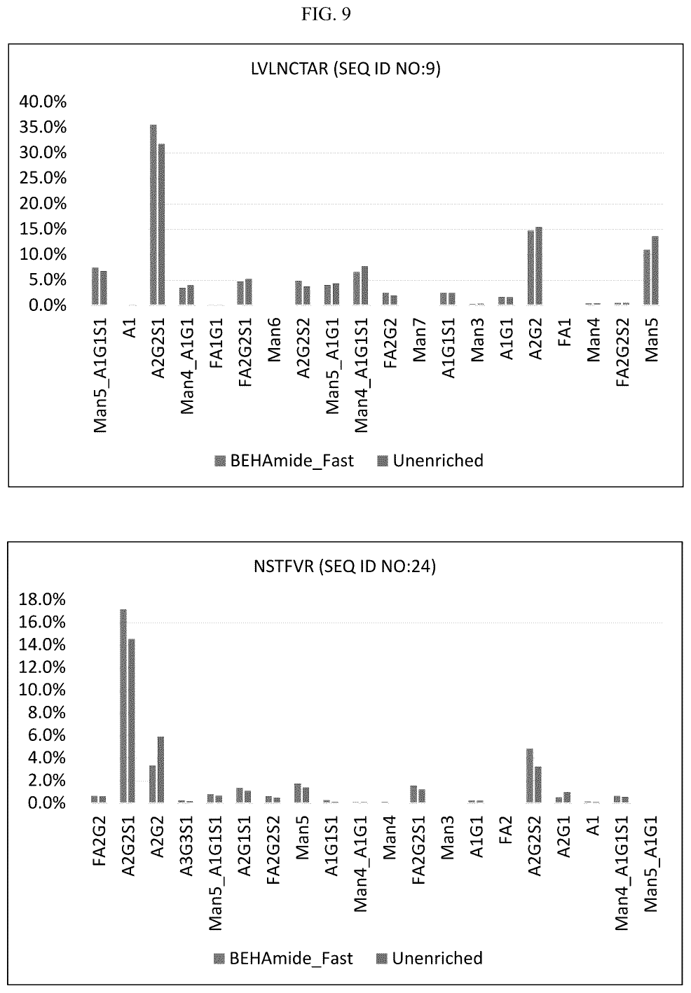

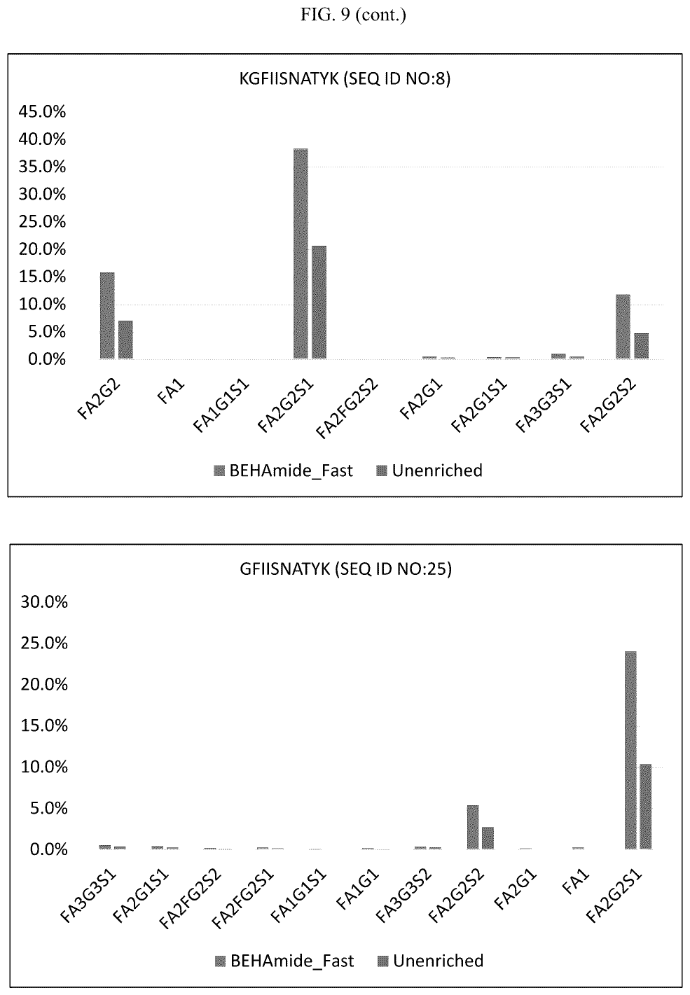

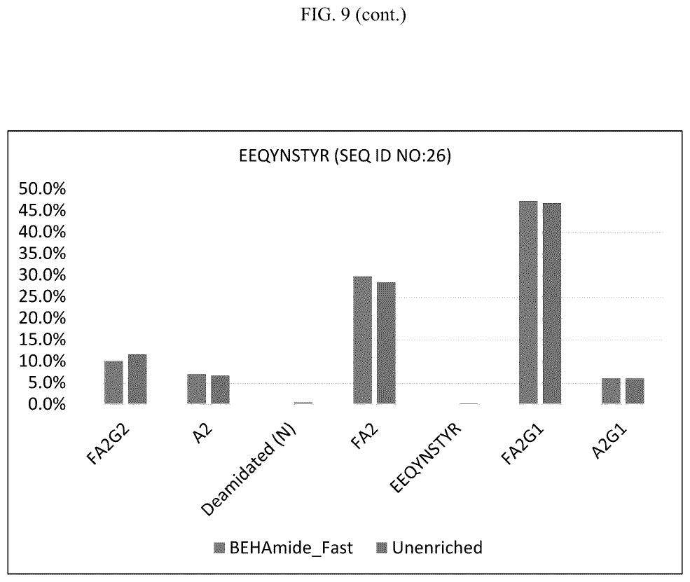

The validation of the glycopeptide enrichment assay was further verified by comparing the mini-trap glycopeptide species identified in enriched versus unenriched samples, as shown in FIG. 9. The relative levels of each glycopeptide species remain about the same after enrichment, demonstrating that the method of the present invention does not alter the relative abundance of each glycopeptide species in a sample. The general increase in glycopeptide levels in enriched samples can be attributed to a lack of interference from high non-glycosylated peptides lost in the HILIC gradient. The confidence of glycopeptide identification increased as the relative levels increased. The population of confidently identified glycopeptides are significantly improved with glycopeptide enrichment.

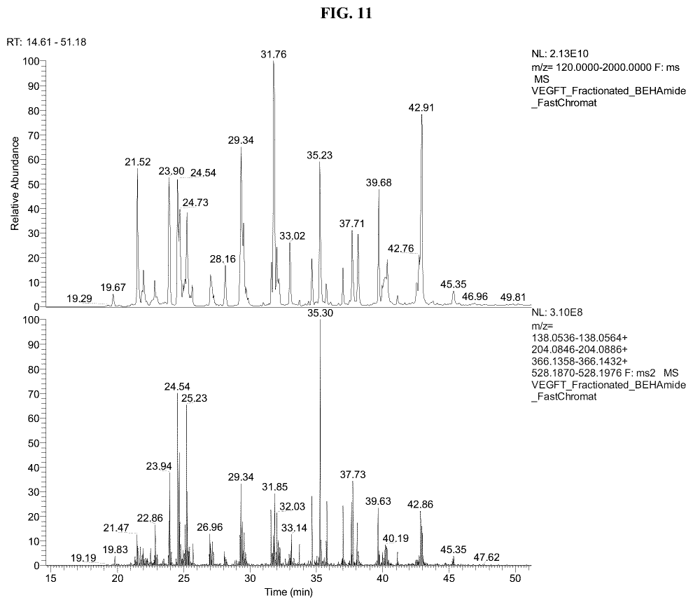

The method of the present invention was further validated using glycopeptide enrichment of a VEGF trap (antibody-receptor fusion) protein. A VEGF trap protein was subjected to enrichment as described in Example 1, and collected glycopeptide enriched fractions were subjected to LC-MS using an Orbitrap Eclipse. FIG. 11 shows a comparison of a total ion current chromatogram (TIC) in the top panel, and signal from oxonium ions in the bottom panel. Oxonium ions, which are a proxy for glycopeptide species, represented the predominate species in the enriched fractions.

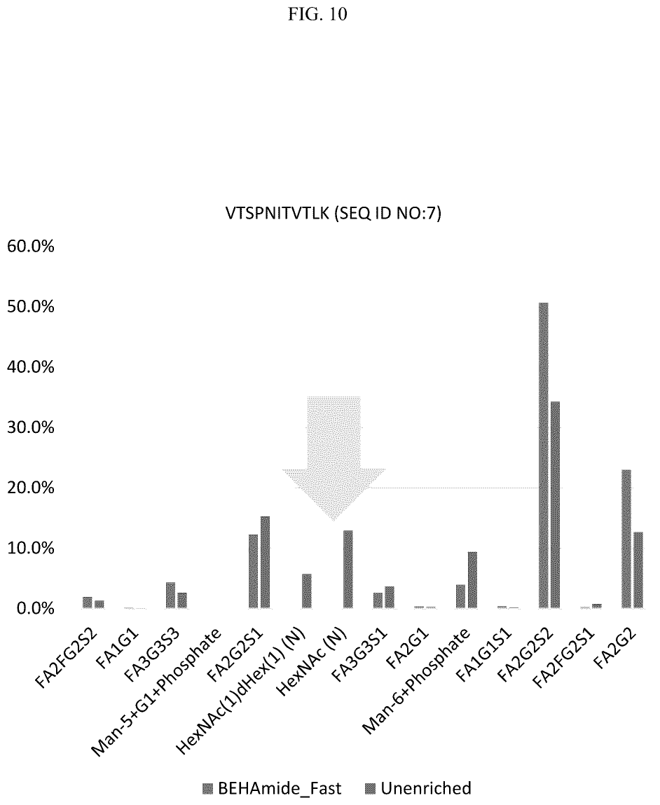

A quantitation of glycopeptide species from the VEGF trap protein identified in enriched versus unenriched samples is shown in FIG. 10. The arrow indicates where a glycopeptide species comprising a small glycan (HexNAc) was lost in the gradient. Glycopeptide species comprising a small glycan may not be enriched by this method because the minor mass increase may not cause a substantive hydrophobic shift, and therefore the hydrophobicity of the small glycan-containing peptides may be similar to non-glycosylated peptides and may be lost during enrichment.

This example demonstrates that the novel method of the present invention, including digestion of a mAb of interest to form peptide fragments and hydrophilic interaction chromatography enrichment utilizing a landmark VDNAL sequence, can identify and characterize glycopeptide species with high sensitivity. Furthermore, this example demonstrates that relative levels of glycopeptides remain the same in enriched versus unenriched samples, validating that the method of the present invention does not change the relative abundances of glycopeptide species.

### Example 3. Optimization of the Mass Spectrometry Method

The method of the present invention was further improved by optimizing the mass spectrometry method used for analysis of enriched glycopeptide samples. MS optimization was carried out to increase the number of glycopeptide species identified. Through optimization of certain acquisition settings, such as increasing AGC target, resolution, and maximum injection time, in comparison to a standard method, additional glycopeptide species were identified in the enriched sample when compared to an unenriched sample. Optimized parameters included an AGC target of 4×10⁴, resolution of 60,000, and maximum injection time of 250 ms, compared to conventional parameters of an AGC target of 1×10⁴, resolution of 15,000, and maximum injection time of 60 ms. Analysis of enriched samples using the optimized acquisition settings led to the production of more peptide spectrum matches (PSMs) compared to a standard method.

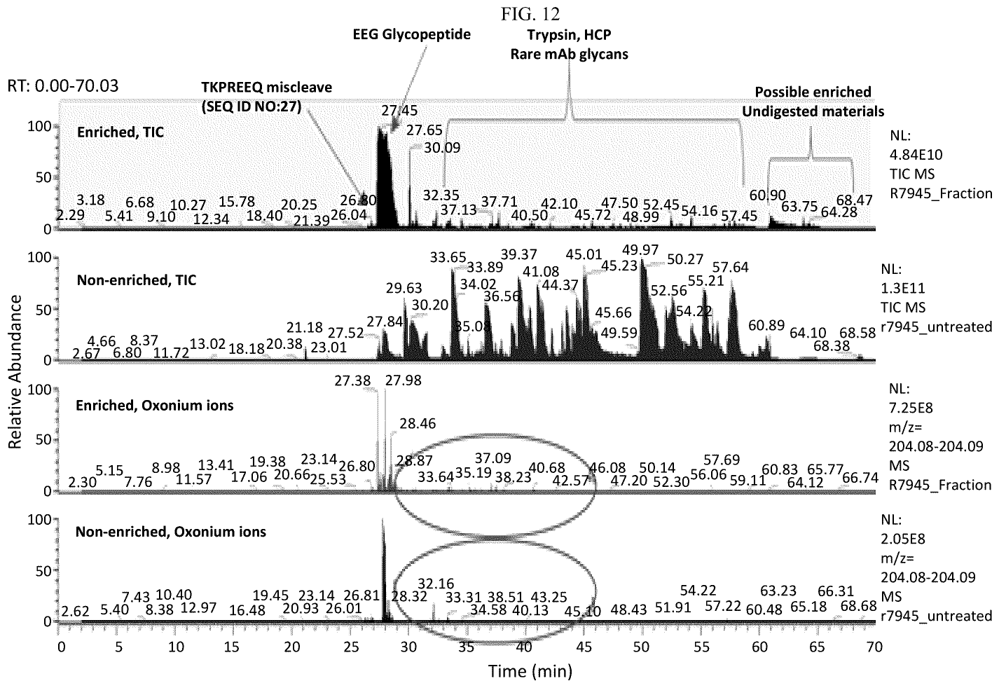

FIG. 12 shows MS spectra for enriched versus non-enriched samples, according to an exemplary embodiment. The total ion current chromatogram (TIC) from the enriched sample has a cleaner profile as compared to the TIC from the non-enriched sample. In addition, the enriched sample has a higher relative abundance of oxonium ions, indicating a higher proportion of glycopeptides.

This example demonstrates that a mAb of interest in a sample can be effectively characterized using a novel method including digestion to form peptide fragments and hydrophilic interaction chromatography enrichment utilizing a landmark VDNAL sequence for characterization. In particular, optimization of mass spectrometry analysis of enriched glycopeptides through increasing an AGC target, resolution, and maximum injection time led to more PSMs and an increased number of glycopeptide species identified.

### Example 4. Utilization of the Byonic Database for Identification of Non-Consensus Glycosylation

This example demonstrates the usefulness of the method of the present invention in identifying and characterizing non-consensus glycosylation in a mAb of interest. Following automated digestion of a mAb and enrichment using HILIC as described in Example 1, the Byonic database was searched for non-consensus glycosylation. In order to identify non-consensus glycosylation in mAb using the Byonic database, three types of glycans were investigated: O-glycans, atypical N-glycans, and glycans associated with a new N-glycan motif resulting from serine to asparagine mutations. Database searching revealed 70 common human O-glycans, 55 common N-glycans and serine to asparagine mutations, such as NQVSLTCLVK (SEQ ID NO: 5)->NQVNLTCLVK (SEQ ID NO: 6). The initial search revealed sequence variant glycopeptide species. This serine to asparagine sequence variant has been previously characterized, however the glycosylation at this mutation has not been previously characterized.

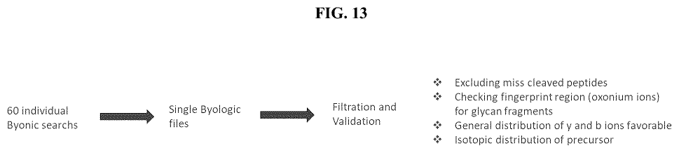

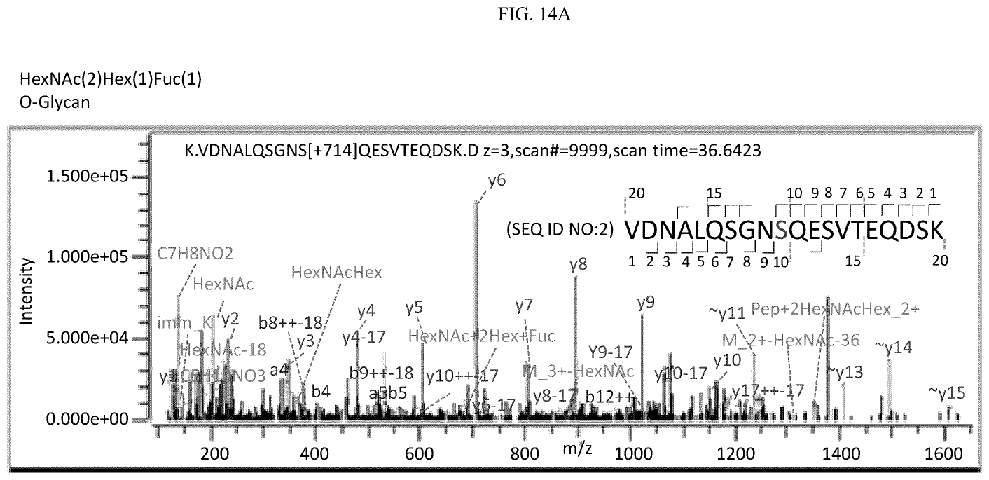

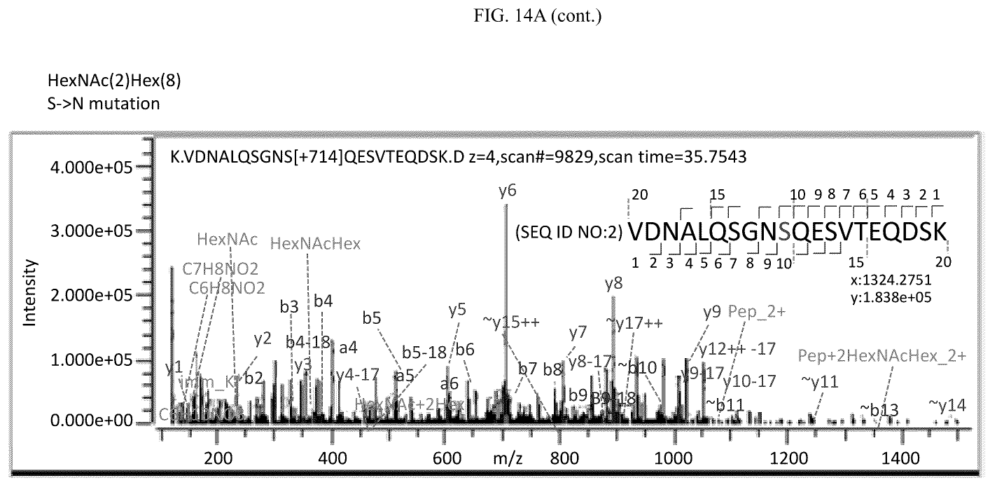

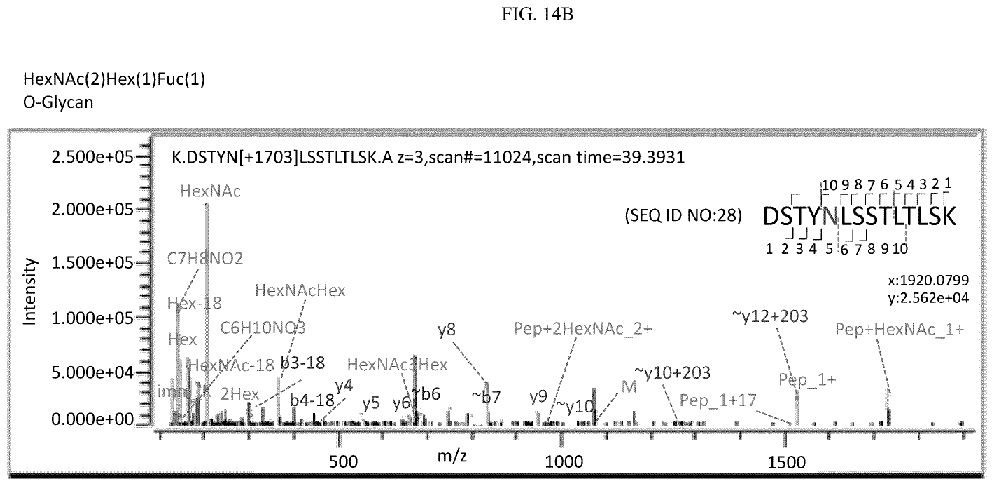

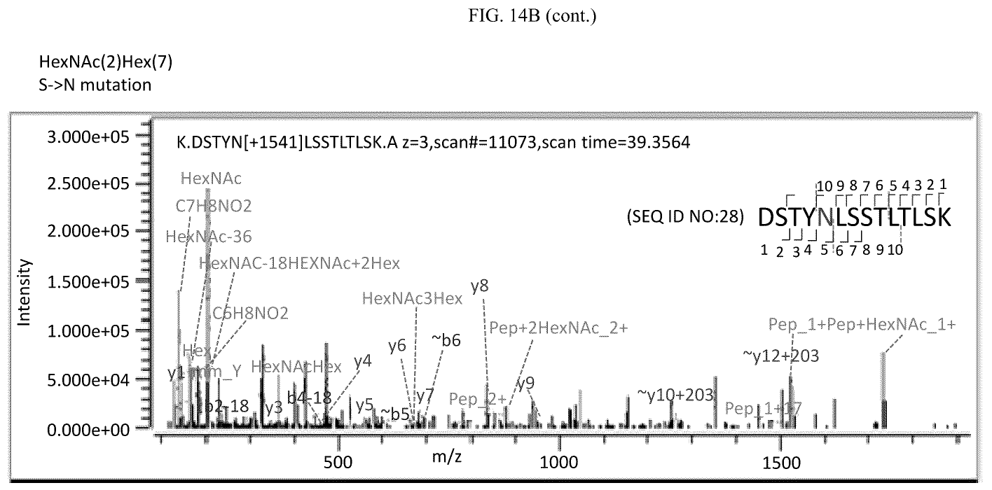

FIG. 13 shows an exemplary workflow of filtering the data in Byonic, commencing with the individual searches, through filtering and validation of the data. Filtration and validation included the steps of excluding miscleaved peptides; checking the fingerprint region (oxonium ions) for glycan fragments; determining if the general distribution of y and b ions was favorable; and determining the isotopic distribution of the precursor. Several non-consensus N-glycosylation sites were observed in CDR1, CDR2, CDR3 and CH1. FIGS. 14A&B show the mass spectra of two exemplary modifications, O-glycan and serine to asparagine mutation, observed in a common region across mAbs. FIG. 14A shows mass spectra for a first exemplary mAb and FIG. 14B shows mass spectra for a second exemplary mAb.

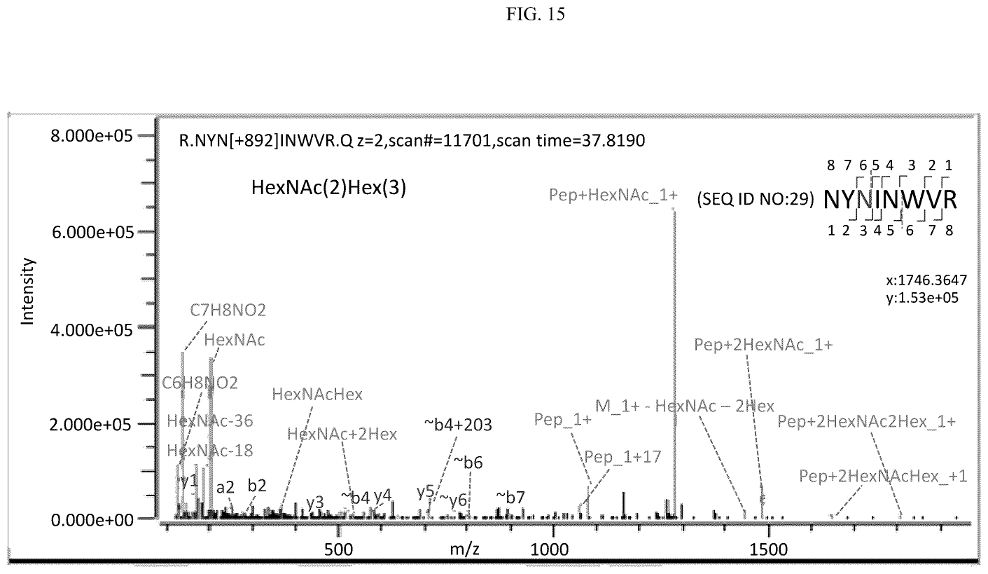

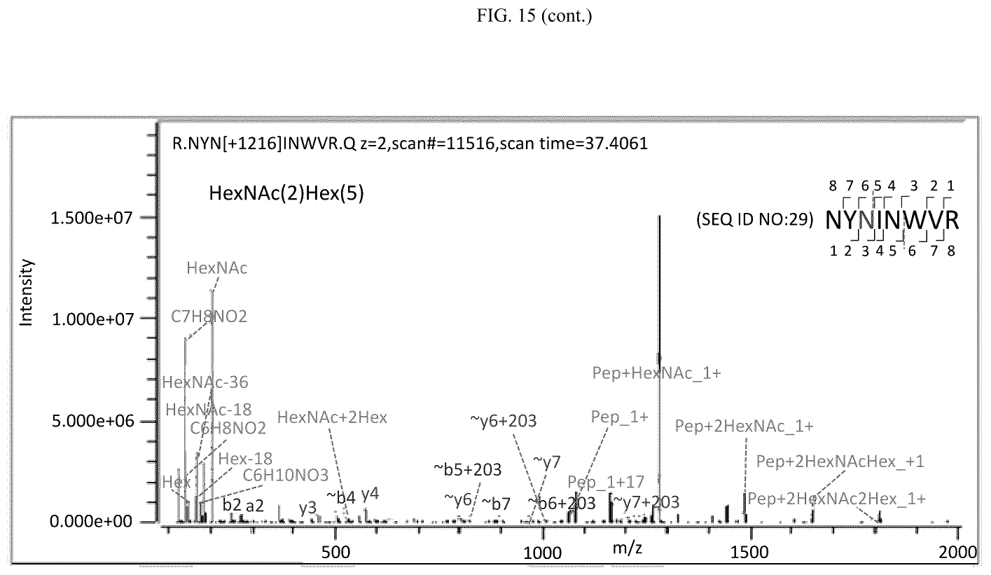

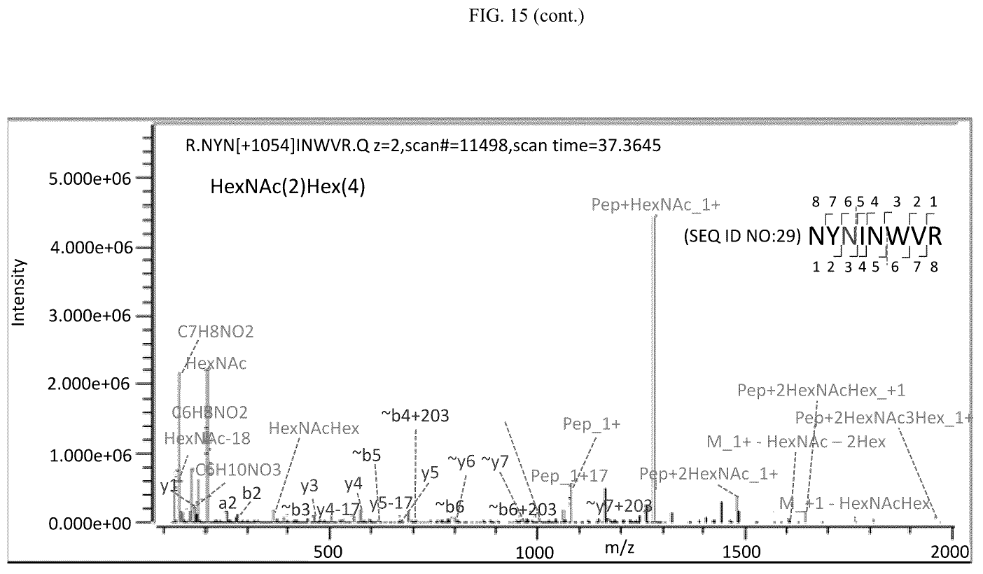

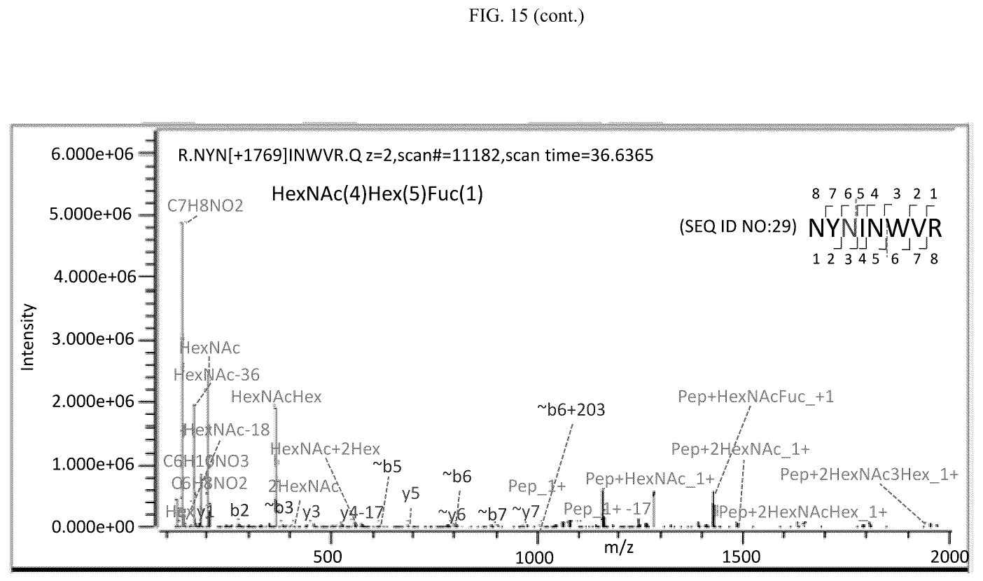

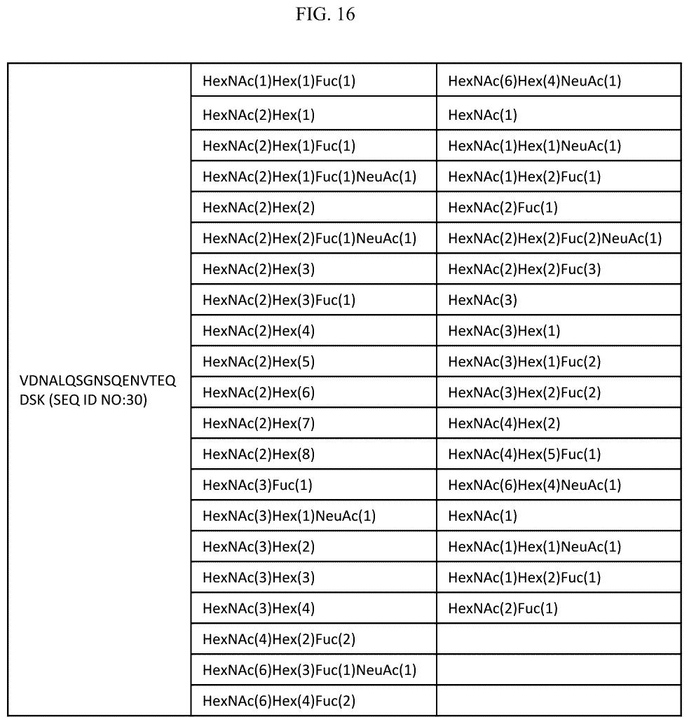

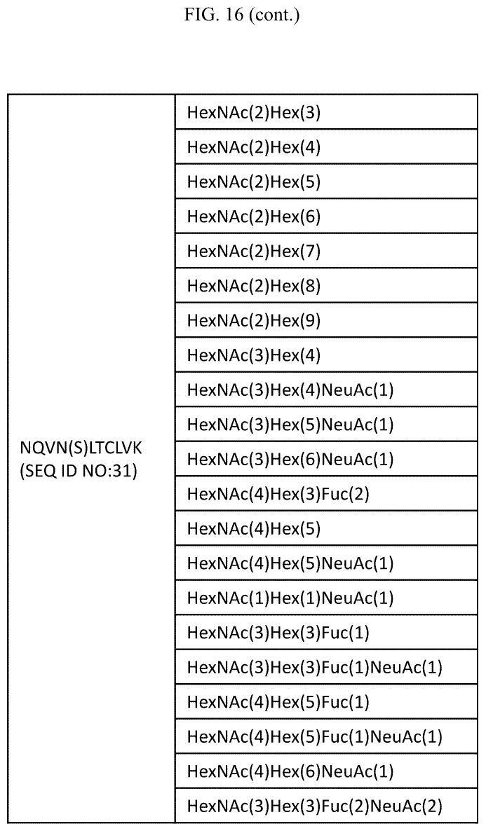

FIG. 15 shows mass spectra from a mAb with a non-canonical N-glycosylation site in the CDR. Multiple glycoforms were identified in the CDR region, demonstrating that the method of the present invention not only allows for characterizing glycosylation by location, but also allows for characterizing the whole glycoform profile of a protein of interest. Enrichment of intact glycopeptides as described in the examples above not only led to the identification of new sites, but identification of the glycoform profiles as well, as shown in FIG. 16.

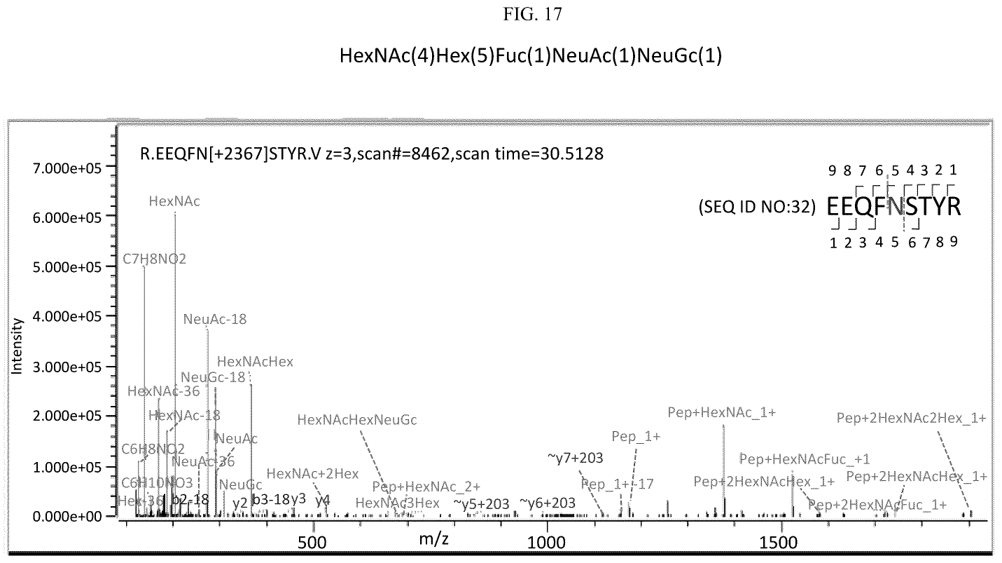

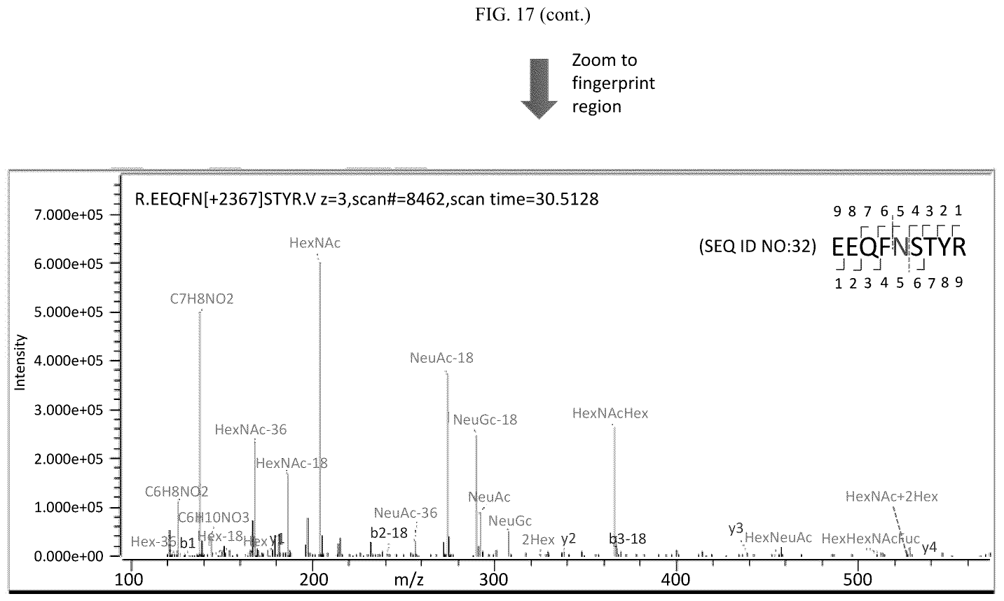

The method of the present invention also identified a non-human glycoform on conventional Fc glycosylation sites, as shown in FIG. 17. The two non-human glycoforms identified are the NeuGc fragments observed in the zoomed in spectrum of the fingerprint region.

This example demonstrates that the novel method of the present invention, including digestion of a mAb of interest to form peptide fragments and hydrophilic interaction chromatography enrichment utilizing a landmark VDNAL sequence, can identify and characterize glycopeptide species with high sensitivity. The utilization of the Byonic database was capable of clearly identifying many characterized and uncharacterized atypical N-glycans, O-glycans, as well as glycans associated with a new N-glycan motif resulting from serine to asparagine mutations. A comprehensive analysis not only of glycosylation sites but of entire glycoform profiles is possible. The identification of multiple glycoforms and glycosites can be used to further understand glycosylation hot spots across various proteins of interest, and what effects may exist in downstream signaling pathways, as well as provide a better understanding of the cause and control of their occurrence.

### Example 5. Quantification for Method Reproducibility

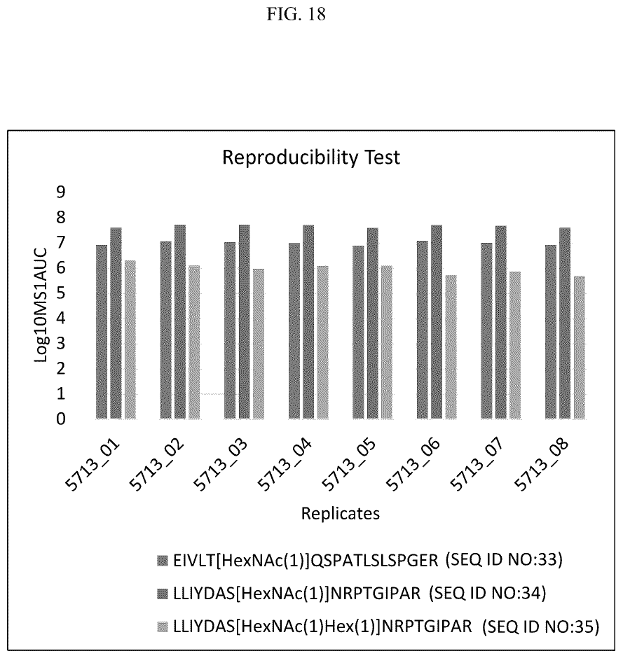

This example demonstrates the reproducibility of the method of the present invention. A representative mAb was subjected to automated digestion and enrichment according to the method described in Example 1 for a total of eight replicates. Three glycopeptides were evaluated, as evidenced in FIG. 18, with each respective peptide exhibiting comparable data across the eight replicates, confirming the reproducibility of the digestion and enrichment methods.

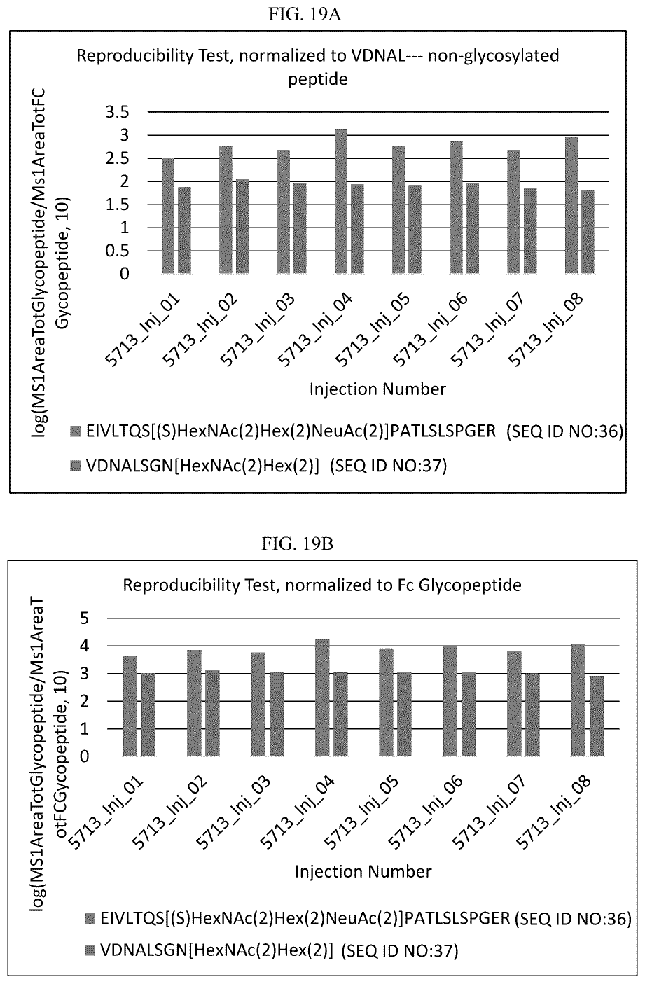

To further test the reproducibility of the method, one peptide generated was normalized to the non-glycosylated VDNAL peptide. In addition, the same peptide was also normalized to the Fc glycopeptide. FIGS. 19A&B show the results of the digestion and enrichment for eight replicates of common glycopeptide sequences that were normalized to a common peptide sequence. FIG. 19A compares a common glycopeptide to the VDNAL, non-glycosylated peptide and FIG. 19B compares a common glycopeptide to the Fc glycopeptide, with each respective peptide exhibiting comparable data across the eight replicates.

This example demonstrates that the novel method of the present invention, including digestion of a mAb of interest to form peptide fragments and hydrophilic interaction chromatography enrichment utilizing a landmark VDNAL sequence, can identify and characterize glycopeptide species with high sensitivity. Furthermore, separate digestions and enrichments for glycopeptide quantification clearly exhibit high reproducibility, confirming the robustness of the present method.

### Example 6. Limited Deglycosylation

Proteins were treated with an endoglycosidase enzyme under conditions that result in limited deglycosylation. The endoglycosidase enzyme, PNGase F, was prepared in 5.75 mU/μL aliquots. 30 μL of Milli-Q water and 20 μL of 10x Glycobuffer was added to 0.15 mL of 500 units/μL of PNGase F to result in a final concentration of 5.75 mU/μL (or 373.75 NEB units). 10 μL aliquots were stored at −20° C. Deglycosylated samples were prepared with a final Tris concentration of 100 mM and an E: S of PNGase F to sample of 1:10 with the final protein concentration being 5 mg/mL. 400 μg of sample was transferred to a vial. The sample was concentrated if the initial concentration was less than 50 mg/mL. 8 μL of Tris-HCl, pH 7.5 was added followed by the addition of 6.95 μL of the PNGase F solution prepared as described above. Milli-Q water was added to a final volume of 80 μL and the sample was incubated at 45° C. for 1 hour. After incubation, the sample was subjected to the enrichment protocol described above.

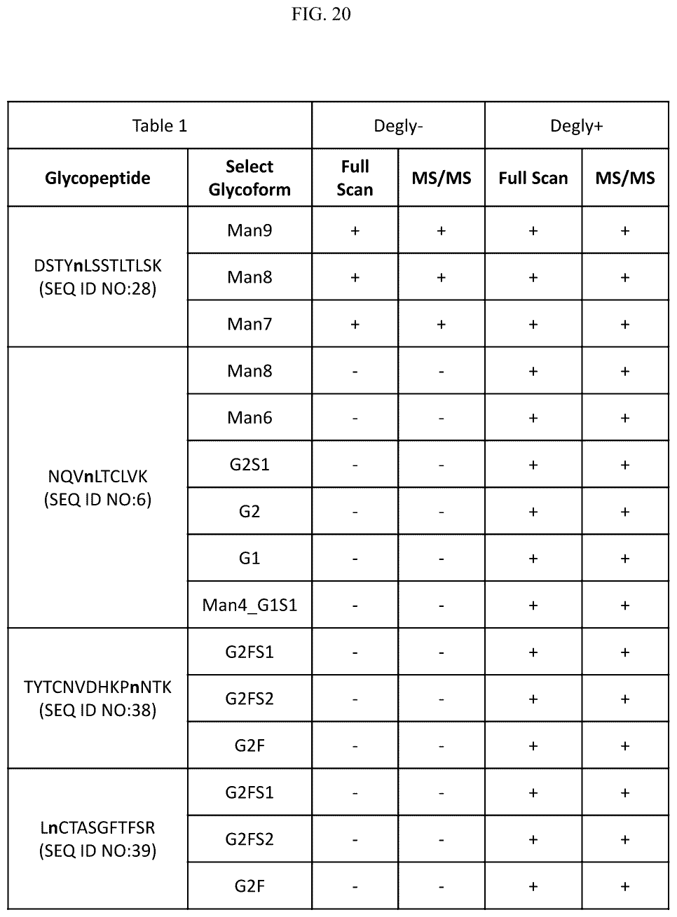

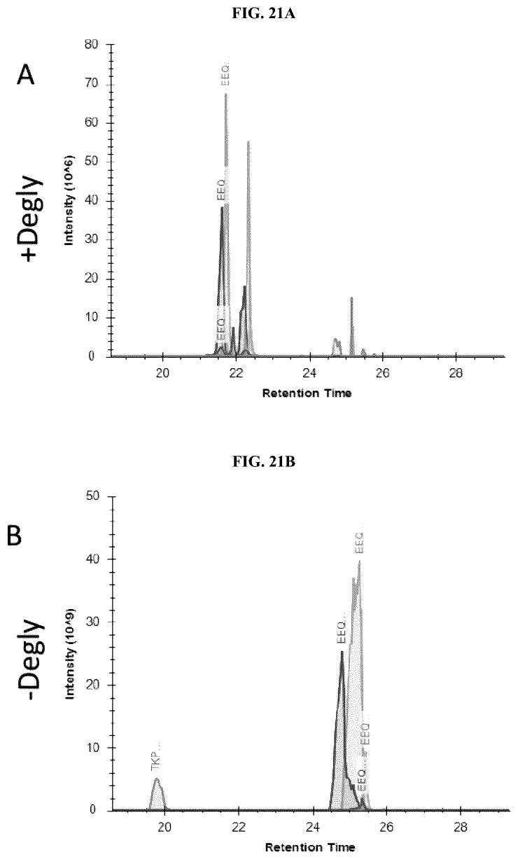

FIG. 20 shows that limited deglycosylation of an exemplary mAb prior to tryptic digestion and glycopeptide enrichment improves identification of non-consensus glycosylation sites and glycoforms containing N-glycan motifs due to S->N sequence variants. Extracted ion chromatograms (XIC) of the top four most abundant glycopeptides from the canonical glycosylation site in Fc region (N297) after HILIC enrichment showed that the level of Fc glycopeptides significantly decreased (by three orders of magnitude) after limited deglycosylation (FIG. 21A) compared with untreated control (FIG. 21B).

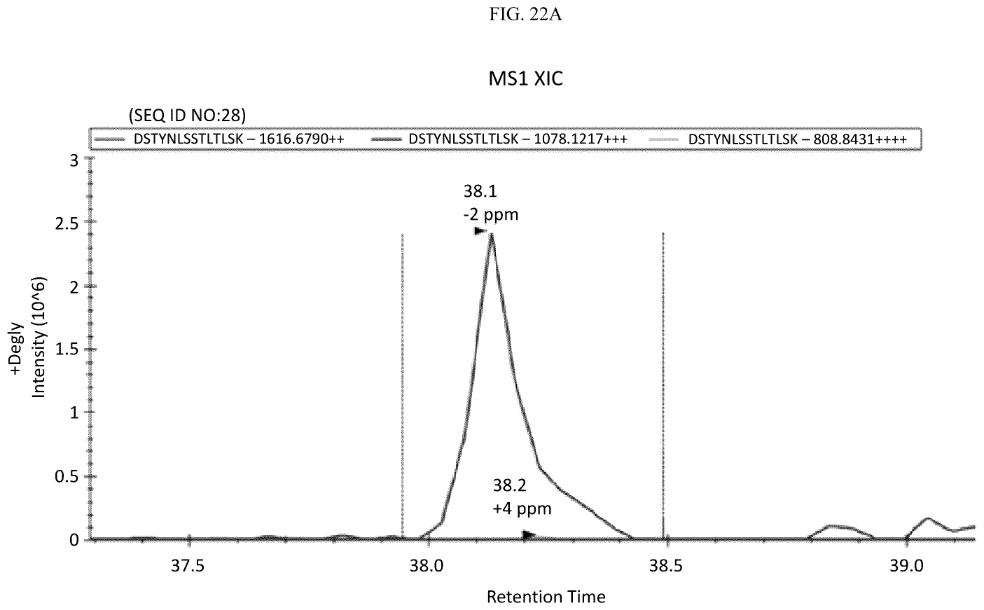

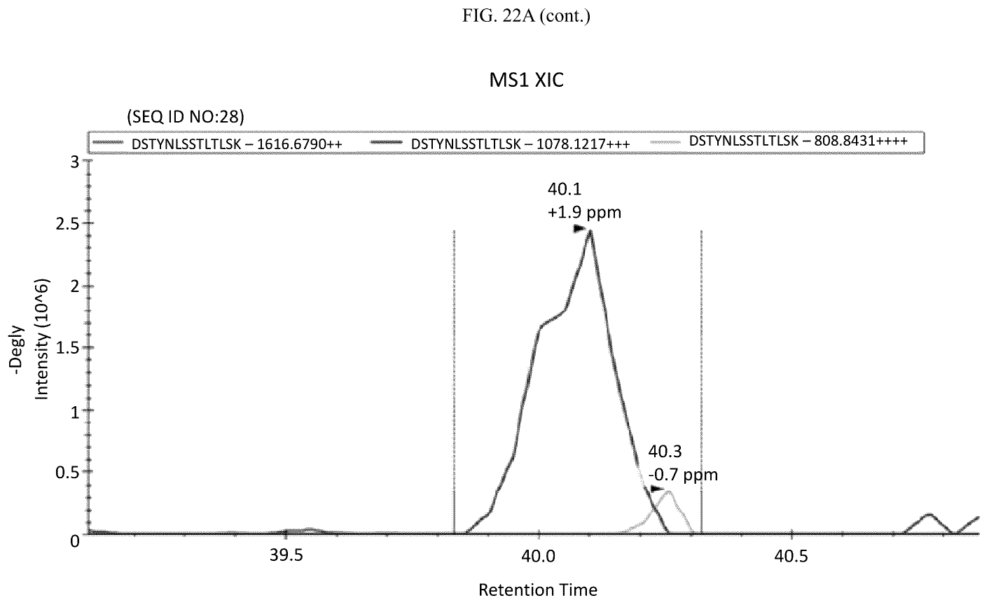

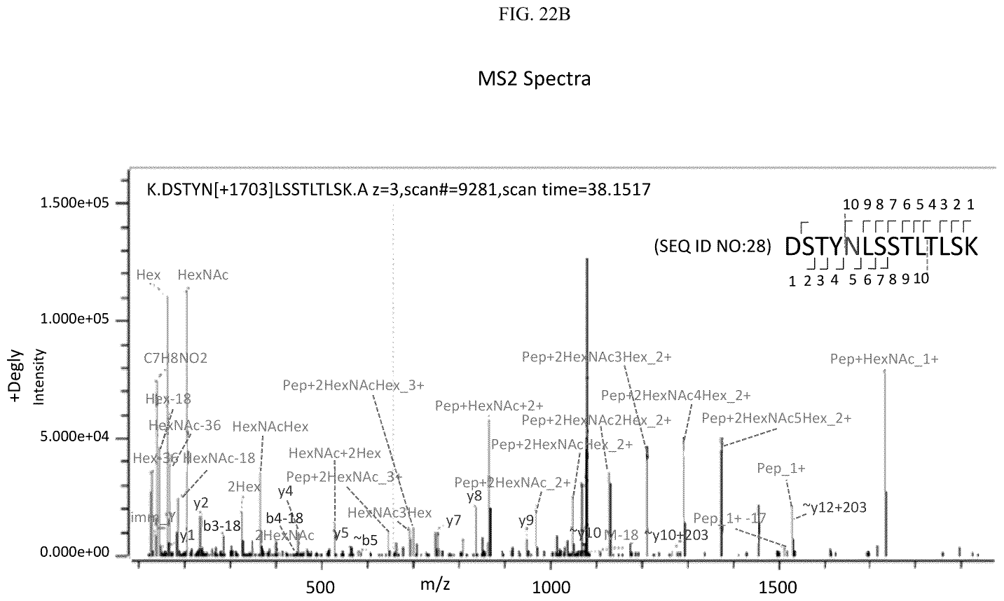

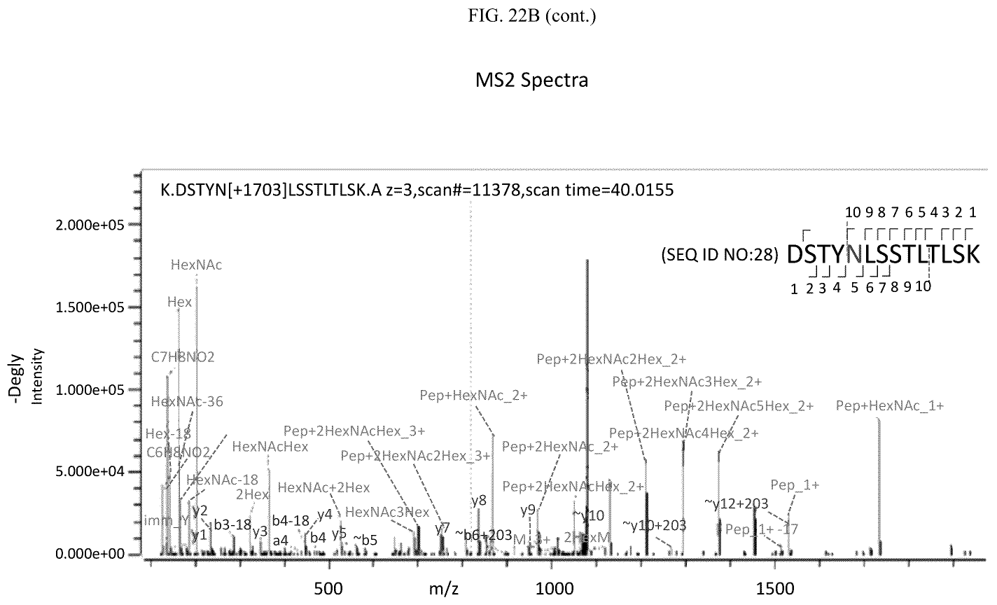

An exemplary glycopeptide from non-canonical glycosylation site detected after HILIC enrichment with and without limited deglycosylation is shown in FIGS. 22A&B. The level of precursor detected in a full scan (FIG. 22A) and the quality of MS/MS scan for glycopeptide identification (FIG. 22B) remain the same. Thus, low level glycosylation from non-canonical sites may not be effectively removed during limited deglycosylation. Therefore, the limited deglycosylation can significantly reduce dynamic range for the presence of both canonical N-glycosylated peptide and non-consensus glycosylation sites in an exemplary mAb.

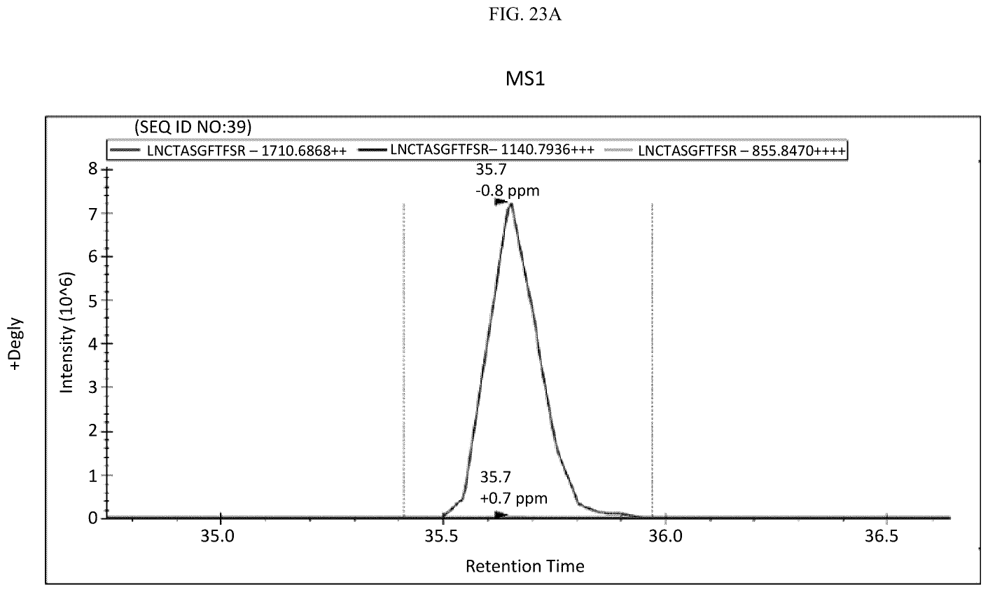

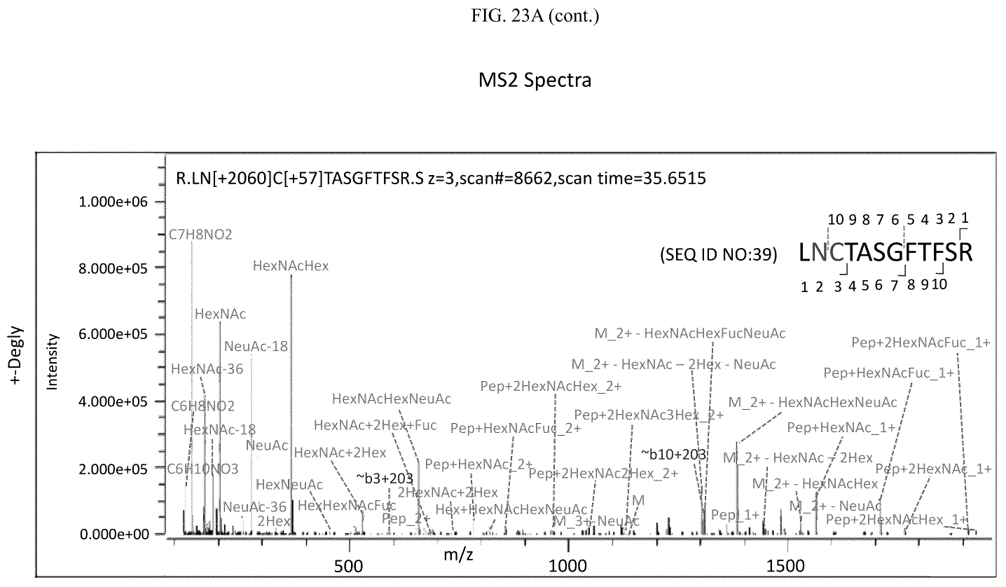

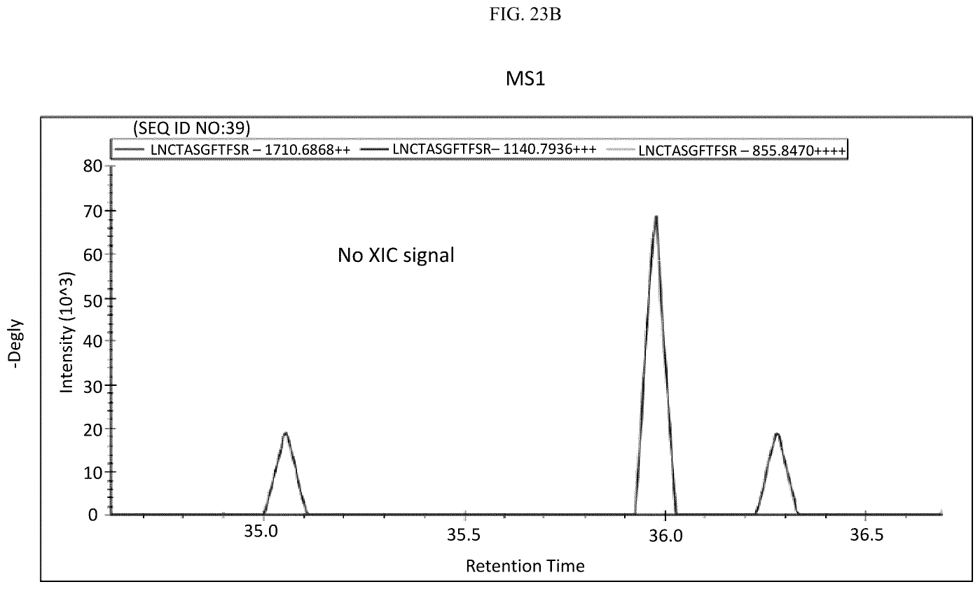

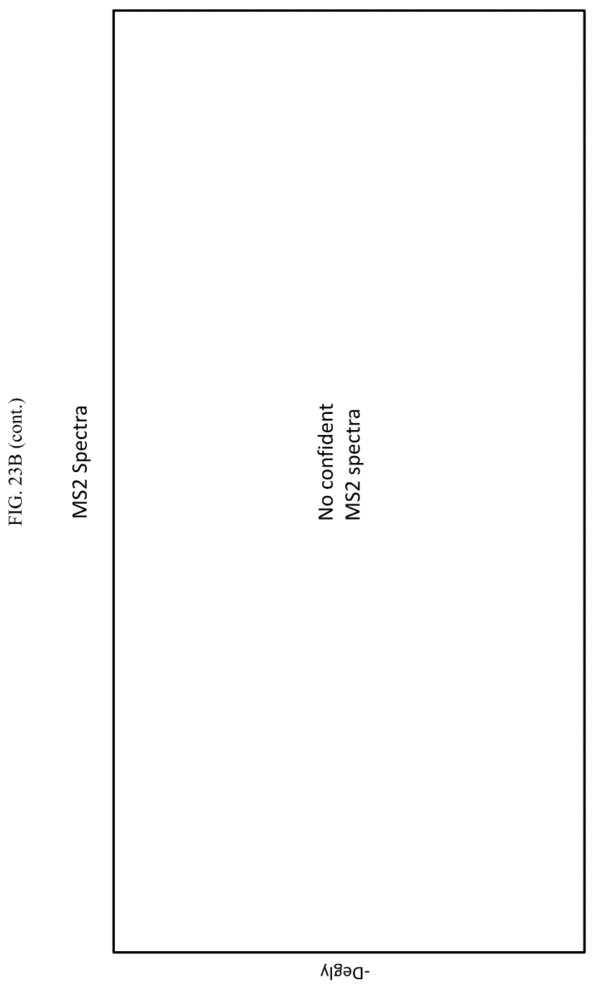

An exemplary N-glycopeptide from a non-consensus glycosylation site was exclusively identified in a sample with limited deglycosylation before tryptic digestion and glycopeptide enrichment (FIG. 23A). No confident spectra from full scan and MS/MS for this glycosylation was detected in untreated sample that still subjected to glycopeptide enrichment (FIG. 23B). This result further confirms that reduced dynamic range due to removal of both regular non-glycosylated peptides (by HILIC enrichment) and N-glycosylated peptide from canonical Fc site (by limited deglycosylation) improve the limit of detection for low glycopeptide in non-consensus sites.

As demonstrated above, the present method allows for a high-throughput validation for characterizing non-consensus N- and O-glycosylations for a mAb of interest. Presented herein is a novel method providing a high-throughput automated approach, involving digestion to form peptide fragments, hydrophilic interaction chromatography for glycopeptide enrichment, and tandem LC-MS/MS for characterizing and identifying glycosylation of proteins and peptides, suitable for development of therapeutic antibodies.

1. A method for characterizing non-consensus glycosylation sites of a protein in a high-throughput process comprising the steps of:
(a) treating said protein with a digestive enzyme to form glycopeptides or a mixture of glycopeptides and non-glycopeptides;
(b) subjecting said glycopeptides or said mixture to hydrophilic interaction liquid chromatography (HILIC) to form enriched glycopeptides; and
(c) subjecting said enriched glycopeptides to liquid-chromatography-tandem mass spectrometry (LC-MS/MS) analysis by adjusting the Automatic Gain Control (AGC) target and/or maximum injection time to characterize intact non-consensus glycosylation sites of said protein.

2. The method of claim 1, wherein said protein is selected from a group consisting of an antibody, a monoclonal antibody, a bispecific antibody, an antibody fragment, an antibody-derived protein, an antigen-binding protein, an antibody-drug conjugate, or a fusion protein.

3. The method of claim 1, wherein said protein is denatured and reduced prior to the digestion of step (a).

4. The method of claim 1, wherein said digestive enzyme is selected from a group consisting of pepsin, trypsin, Tryp-N, chymotrypsin, Lys-N, Lys-C, Asp-N, Arg-C, Glu-C, papain, IdeS, and variants thereof.

5. The method of claim 1, wherein said treating said protein with a digestive enzyme is automated.

6. The method of claim 1, wherein said liquid chromatography is automated.

7. The method of claim 1, wherein said mass spectrometer is an electrospray ionization mass spectrometer, nano-electrospray ionization mass spectrometer, or an Orbitrap-based mass spectrometer, wherein said mass spectrometer is coupled to said liquid chromatography system.

8. The method of claim 1, wherein said AGC target is about of 4×10⁴ and the maximum injection time is about 250 ms.

9. The method of claim 1, wherein said HILIC step further comprises comparing said glycopeptides to a landmark VDNAL-peak as a divider between non-glycosylated and glycosylated peptides as related to retention time.

10. The method of claim 1, wherein non-consensus glycosylations are selected from the group consisting of N-glycosylation, O-glycosylation and sequence variants of S->N mutations.

11. The method of claim 1, wherein said non-consensus glycosylations are identified by comparison to a database of known non-consensus glycosylations.

12. The method of claim 11, wherein said database is the Byonic database.

13. The method of claim 1 further comprising treating said protein with an endoglycosidase enzyme prior to the digestion of step (a).

14. The method of claim 13, wherein the endoglycosidase enzyme is selected from the group consisting of PNGase F, endoglycosidase F1, endoglycosidase F2, endoglycosidase F3, endoglycosidase H, O-glycosidase, Endo-B-Galactosidase and combinations thereof.

15. The method of claim 14, wherein the endoglycosidase enzyme is PNGase F.

16. A method for characterizing glycosylation sites of a protein in a high-throughput process comprising the steps of:
(a) treating said protein with a digestive enzyme to form glycopeptides or a mixture of glycopeptides and non-glycopeptides;
(b) subjecting said glycopeptides or said mixture to a first liquid chromatography step to form enriched glycopeptides; and
(c) subjecting said enriched glycopeptides to a second liquid chromatography step coupled to tandem mass spectrometry (LC-MS/MS) analysis by adjusting the AGC target and/or maximum injection time to characterize glycosylation sites of said protein.

17. The method of claim 16, wherein said protein is selected from a group consisting of an antibody, a monoclonal antibody, a bispecific antibody, an antibody fragment, an antibody-derived protein, an antigen-binding protein, an antibody-drug conjugate, or a fusion protein.

18. The method of claim 16, wherein said protein is denatured and reduced prior to the digestion of step (a).

19. The method of claim 16, wherein said digestive enzyme is selected from a group consisting of pepsin, trypsin, Tryp-N, chymotrypsin, Lys-N, Lys-C, Asp-N, Arg-C, Glu-C, papain, IdeS, and variants thereof.

20. The method of claim 16, wherein said treating said protein with a digestive enzyme is automated.

21. The method of claim 16, wherein said first liquid chromatography step and/or said second liquid chromatography step comprises reversed phase liquid chromatography, ion exchange chromatography, anion exchange chromatography, weak cation exchange chromatography, strong cation exchange chromatography, size exclusion chromatography, affinity chromatography, hydrophobic interaction chromatography, hydrophilic interaction liquid chromatography (HILIC), mixed-mode chromatography, or a combination thereof.

22. The method of claim 20, wherein said first liquid chromatography step comprises HILIC.

23. The method of claim 16, wherein said first liquid chromatography step and/or said second liquid chromatography step is automated.

24. The method of claim 16, wherein said mass spectrometer is an electrospray ionization mass spectrometer, nano-electrospray ionization mass spectrometer, or an Orbitrap-based mass spectrometer, wherein said mass spectrometer is coupled to said liquid chromatography system.

25. The method of claim 16, wherein said first liquid chromatography step further comprises comparing said glycopeptides to a landmark VDNAL-peak as a divider between non-glycosylated and glycosylated peptides as related to retention time.

26. The method of claim 16, wherein said glycosylations are identified by comparison to a database of known non-consensus glycosylations.

27. The method of claim 26, wherein said database is the Byonic database.

28. The method of claim 16, wherein glycosylations identified are non-consensus glycosylations.

29. The method of claim 28, wherein said non-consensus glycosylations are selected from the group consisting of N-glycosylation, O-glycosylation and sequence variants of S->N mutations.

30. The method of claim 16, wherein said AGC target is about 4×10⁴ and the maximum injection time is about 250 ms.

31. The method of claim 16 further comprising treating said protein with an endoglycosidase enzyme prior to the digestion of step (a).

32. The method of claim 31, wherein the endoglycosidase enzyme is selected from the group consisting of PNGase F, endoglycosidase F1, endoglycosidase F2, endoglycosidase F3, endoglycosidase H, O-glycosidase, Endo-B-Galactosidase and combinations thereof.

33. The method of claim 32, wherein the endoglycosidase enzyme is PNGase F.

34.-65. (canceled)
